(19) United States
(12) Patent Application Publication
(10) Pub. No.: US 2009/0098115 A1
(43) Pub. Date: Apr. 16, 2009

(54) Cell lines and animal models of HER2 expressing tumors

(76) Inventors: Lisa Michele Crocker, San Bruno, CA (US); Sarajane Ross, San Francisco, CA (US); Gail Dianne Phillips, San Carlos, CA (US)

Correspondence Address: GOODWIN PROCTER LLP, 135 COMMONWEALTH DRIVE, MENLO PARK, CA, 94025, US

(21) Appl. No.: 11/875,251

(22) Filed: Oct. 19, 2007

Related U.S. Application Data
(60) Provisional application No. 60/894,163, filed on Mar. 9, 2007.
(60) Provisional application No. 60/862,268, filed on Oct. 20, 2006.

(51) Int. Cl. A61K 39/395; C12N 5/06; A01K 67/027; A61K 38/02; C12Q 1/02

(52) U.S. Cl. 424/133.1; 435/325; 800/10; 800/3; 435/29; 424/174.1; 514/2; 424/178.1

(57) ABSTRACT

The present invention concerns cell lines and animal models of HER2-expressing tumors. In particular, the invention concerns cell lines and animal models of HER2-expressing tumors not responding or responding poorly to treatment with trastuzumab (HERCEPTIN®, Genentech, Inc.). The animal models and cell lines of the invention are useful for evaluating the efficacy of various therapeutic approaches for the treatment of such tumors.

### RELATED APPLICATIONS

The application claims the benefit under 35 U.S.C. §119 to U.S. Provisional Patent Application Ser. No. 60/894,163, filed Mar. 9, 2007 and Ser. No. 60/862,268, filed Oct. 20, 2006, the entire disclosures of which are hereby incorporated in their entirety by reference.

### FIELD OF THE INVENTION

The present invention concerns cell lines and animal models of HER2-expressing tumors. In particular, the invention concerns cell lines and animal models of HER2-expressing tumors not responding or responding poorly to treatment with trastuzumab (HERCEPTIN®, Genentech, Inc.). The animal models and cell lines of the invention are useful for evaluating the efficacy of various therapeutic approaches for the treatment of such tumors.

### BACKGROUND OF THE INVENTION

The HER family of receptor tyrosine kinases are important mediators of cell growth, differentiation and survival. The receptor family includes four distinct members including epidermal growth factor receptor (EGFR, ErbB1, or HER1), HER2 (ErbB2 or p185^{neu}), HER3 (ErbB3) and HER4 (ErbB4 or tyro2).

EGFR, encoded by the erbB1 gene, has been causally implicated in human malignancy. In particular, increased expression of EGFR has been observed in breast, bladder, lung, head, neck and stomach cancer as well as glioblastomas. Increased EGFR receptor expression is often associated with increased production of the EGFR ligand, transforming growth factor alpha (TGF-α), by the same tumor cells resulting in receptor activation by an autocrine stimulatory pathway. Baselga and Mendelsohn Pharmac. Ther. 64:127-154 (1994). Monoclonal antibodies directed against the EGFR or its ligands, TGF-α and EGF, have been evaluated as therapeutic agents in the treatment of such malignancies. See, e.g., Baselga and Mendelsohn., supra; Masui et al. Cancer Research 44:1002-1007 (1984); and Wu et al. J. Clin. Invest. 95:1897-1905 (1995).

The second member of the HER family, p185^{neu}, was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis (Slamon et al., Science, 235:177-182 (1987); Slamon et al., Science, 244:707-712 (1989); and U.S. Pat. No. 4,968,603). To date, no point mutation analogous to that in the neu proto-oncogene has been reported for human tumors. Overexpression of HER2 (frequently but not uniformly due to gene amplification) has also been observed in other carcinomas including carcinomas of the stomach, endometrium, salivary gland, lung, kidney, colon, thyroid, pancreas and bladder. See, among others, King et al., Science, 229:974 (1985); Yokota et al., Lancet: 1:765-767 (1986); Fukushige et al., Mol Cell Biol., 6:955-958 (1986); Guerin et al., Oncogene Res., 3:21-31 (1988); Cohen et al., Oncogene, 4:81-88 (1989); Yonemura et al., Cancer Res., 51:1034 (1991); Borst et al., Gynecol. Oncol., 38:364 (1990); Weiner et al., Cancer Res., 50:421-425 (1990); Kern et al., Cancer Res., 50:5184 (1990); Park et al., Cancer Res., 49:6605 (1989); Zhau et al., Mol. Carcinog., 3:254-257 (1990); Aasland et al. Br. J. Cancer 57:358-363 (1988); Williams et al. Pathobiology 59:46-52 (1991); and McCann et al., Cancer, 65:88-92 (1990). HER2 may be overexpressed in prostate cancer (Gu et al. Cancer Lett. 99:185-9 (1996); Ross et al. Hum. Pathol. 28:827-33 (1997); Ross et al. Cancer 79:2162-70 (1997); and Sadasivan et al. J. Urol. 150:126-31 (1993)).

Antibodies directed against the rat p185^{neu} and human HER2 protein products have been described.

Drebin and colleagues have raised antibodies against the rat neu gene product, p185^{neu} See, for example, Drebin et al., Cell 41:695-706 (1985); Myers et al., Meth. Enzym. 198:277-290 (1991); and WO94/22478. Drebin et al. Oncogene 2:273-277 (1988) report that mixtures of antibodies reactive with two distinct regions of p185^{neu} result in synergistic anti-tumor effects on neu-transformed NIH-3T3 cells implanted into nude mice. See also U.S. Pat. No. 5,824,311 issued Oct. 20, 1998.

Hudziak et al., Mol. Cell. Biol. 9(3):1165-1172 (1989) describe the generation of a panel of HER2 antibodies which were characterized using the human breast tumor cell line SK-BR-3. Relative cell proliferation of the SK-BR-3 cells following exposure to the antibodies was determined by crystal violet staining of the monolayers after 72 hours. Using this assay, maximum inhibition was obtained with the antibody called 4D5 which inhibited cellular proliferation by 56%. Other antibodies in the panel reduced cellular proliferation to a lesser extent in this assay. The antibody 4D5 was further found to sensitize HER2-overexpressing breast tumor cell lines to the cytotoxic effects of TNF-α. See also U.S. Pat. No. 5,677,171 issued Oct. 14, 1997. The HER2 antibodies discussed in Hudziak et al. are further characterized in Fendly et al. Cancer Research 50:1550-1558 (1990); Kotts et al. In Vitro 26(3):59A (1990); Sarup et al. Growth Regulation 1:72-82 (1991); Shepard et al. J. Clin. Immunol. 11(3):117-127 (1991); Kumar et al. Mol. Cell. Biol. 11(2):979-986 (1991); Lewis et al. Cancer Immunol. Immunother. 37:255-263 (1993); Pietras et al. Oncogene 9:1829-1838 (1994); Vitetta et al. Cancer Research 54:5301-5309 (1994); Sliwkowski et al. J. Biol. Chem. 269(20):14661-14665 (1994); Scott et al. J. Biol. Chem. 266:14300-5 (1991); D'souza et al. Proc. Natl. Acad. Sci. 91:7202-7206 (1994); Lewis et al. Cancer Research 56:1457-1465 (1996); and Schaefer et al. Oncogene 15:1385-1394 (1997).

A recombinant humanized version of the murine HER2 antibody 4D5 (huMAb4D5-8, rhuMAb HER2, trastuzumab or HERCEPTIN®; U.S. Pat. No. 5,821,337) is clinically active in patients with HER2-overexpressing metastatic breast cancers that have received extensive prior anti-cancer therapy (Baselga et al., J. Clin. Oncol. 14:737-744 (1996)). Trastuzumab received marketing approval from the Food and Drug Administration Sep. 25, 1998 for the treatment of patients with metastatic breast cancer whose tumors overexpress the HER2 protein.

Other HER2 antibodies with various properties have been described in Tagliabue et al. Int. J. Cancer 47:933-937 (1991); McKenzie et al. Oncogene 4:543-548 (1989); Maier et al. Cancer Res. 51:5361-5369 (1991); Bacus et al. Molecular Carcinogenesis 3:350-362 (1990); Stancovski et al. PNAS (USA) 88:8691-8695 (1991); Bacus et al. Cancer Research 52:2580-2589 (1992); Xu et al. Int. J. Cancer 53:401-408 (1993); WO94/00136; Kasprzyk et al. Cancer Research 52:2771-2776 (1992); Hancock et al. Cancer Res. 51:4575-4580 (1991); Shawver et al. Cancer Res. 54:1367-1373 (1994); Arteaga et al. Cancer Res. 54:3758-3765 (1994); Harwerth et al. J. Biol. Chem. 267:15160-15167 (1992); U.S. Pat. No. 5,783,186; and Klapper et al. Oncogene 14:2099-2109 (1997).

Homology screening has resulted in the identification of two other HER receptor family members; HER3 (U.S. Pat. Nos. 5,183,884 and 5,480,968 as well as Kraus et al. PNAS (USA) 86:9193-9197 (1989)) and HER4 (EP Pat Appln No 599,274; Plowman et al., Proc. Natl. Acad. Sci. USA, 90:1746-1750 (1993); and Plowman et al., Nature, 366:473-475 (1993)). Both of these receptors display increased expression on at least some breast cancer cell lines.

The HER receptors are generally found in various combinations in cells and heterodimerization is thought to increase the diversity of cellular responses to a variety of HER ligands (Earp et al. Breast Cancer Research and Treatment 35: 115-132 (1995)). EGFR is bound by six different ligands; epidermal growth factor (EGF), transforming growth factor alpha (TGF-α), amphiregulin, heparin binding epidermal growth factor (HB-EGF), betacellulin and epiregulin (Groenen et al. Growth Factors 11:235-257 (1994)). A family of heregulin proteins resulting from alternative splicing of a single gene are ligands for HER3 and HER4. The heregulin family includes alpha, beta and gamma heregulins (Holmes et al., Science, 256:1205-1210 (1992); U.S. Pat. No. 5,641,869; and Schaefer et al. Oncogene 15:1385-1394 (1997)); neu differentiation factors (NDFs), glial growth factors (GGFs); acetylcholine receptor inducing activity (ARIA); and sensory and motor neuron derived factor (SMDF). For a review, see Groenen et al. Growth Factors 11:235-257 (1994); Lemke, G. Molec. & Cell. Neurosci. 7:247-262 (1996) and Lee et al. Pharm. Rev. 47:51-85 (1995). Recently three additional HER ligands were identified; neuregulin-2 (NRG-2) which is reported to bind either HER3 or HER4 (Chang et al. Nature 387 509-512 (1997); and Carraway et al Nature 387:512-516 (1997)); neuregulin-3 which binds HER4 (Zhang et al. PNAS (USA) 94(18):9562-7 (1997)); and neuregulin-4 which binds HER4 (Harari et al. Oncogene 18:2681-89 (1999)) HB-EGF, betacellulin and epiregulin also bind to HER4.

While EGF and TGFα do not bind HER2, EGF stimulates EGFR and HER2 to form a heterodimer, which activates EGFR and results in transphosphorylation of HER2 in the heterodimer. Dimerization and/or transphosphorylation appears to activate the HER2 tyrosine kinase. See Earp et al., supra. Likewise, when HER3 is co-expressed with HER2, an active signaling complex is formed and antibodies directed against HER2 are capable of disrupting this complex (Sliwkowski et al., J. Biol. Chem., 269(20):14661-14665 (1994)). Additionally, the affinity of HER3 for heregulin (HRG) is increased to a higher affinity state when co-expressed with HER2. See also, Levi et al., Journal of Neuroscience 15: 1329-1340 (1995); Morrissey et al., Proc. Natl. Acad. Sci. USA 92: 1431-1435 (1995); and Lewis et al., Cancer Res., 56:1457-1465 (1996) with respect to the HER2-HER3 protein complex. HER4, like HER3, forms an active signaling complex with HER2 (Carraway and Cantley, Cell 78:5-8 (1994)).

Patent publications related to HER antibodies include: U.S. Pat. No. 5,677,171, U.S. Pat. No. 5,720,937, U.S. Pat. No. 5,720,954, U.S. Pat. No. 5,725,856, U.S. Pat. No. 5,770,195, U.S. Pat. No. 5,772,997, U.S. Pat. No. 6,165,464, U.S. Pat. No. 6,387,371, U.S. Pat. No. 6,399,063, US2002/0192212A1, U.S. Pat. No. 6,015,567, U.S. Pat. No. 6,333,169, U.S. Pat. No. 4,968,603, U.S. Pat. No. 5,821,337, U.S. Pat. No. 6,054,297, U.S. Pat. No. 6,407,213, U.S. Pat. No. 6,719,971, U.S. Pat. No. 6,800,738, US2004/0236078A1, U.S. Pat. No. 5,648,237, U.S. Pat. No. 6,267,958, U.S. Pat. No. 6,685,940, U.S. Pat. No. 6,821,515, WO98/17797, U.S. Pat. No. 6,127,526, U.S. Pat. No. 6,333,398, U.S. Pat. No. 6,797,814, U.S. Pat. No. 6,339,142, U.S. Pat. No. 6,417,335, U.S. Pat. No. 6,489,447, WO99/31140, US2003/0147884A1, US2003/0170234A1, US2005/0002928A1, U.S. Pat. No. 6,573,043, US2003/0152987A1, WO99/48527, US2002/0141993A1, WO01/00245, US2003/0086924, US2004/0013667A1, WO00/69460, WO01/00238, WO01/15730, U.S. Pat. No. 6,627,196B1, U.S. Pat. No. 6,632,979B1, WO01/00244, US2002/0090662A1, WO01/89566, US2002/0064785, US2003/0134344, WO 04/24866, US2004/0082047, US2003/0175845A1, WO03/087131, US2003/0228663, WO2004/008099A2, US2004/0106161, WO2004/048525, US2004/0258685A1, U.S. Pat. No. 5,985,553, U.S. Pat. No. 5,747,261, U.S. Pat. No. 4,935,341, U.S. Pat. No. 5,401,638, U.S. Pat. No. 5,604,107, WO 87/07646, WO 89/10412, WO 91/05264, EP 412,116 B1, EP 494,135 B1, U.S. Pat. No. 5,824,311, EP 444,181 B1, EP 1,006,194 A2, US 2002/0155527A1, WO 91/02062, U.S. Pat. No. 5,571,894, U.S. Pat. No. 5,939,531, EP 502,812 B1, WO 93/03741, EP 554,441 B1, EP 656,367 A1, U.S. Pat. No. 5,288,477, U.S. Pat. No. 5,514,554, U.S. Pat. No. 5,587,458, WO 93/12220, WO 93/16185, U.S. Pat. No. 5,877,305, WO 93/21319, WO 93/21232, U.S. Pat. No. 5,856,089, WO 94/22478, U.S. Pat. No. 5,910,486, U.S. Pat. No. 6,028,059, WO 96/07321, U.S. Pat. No. 5,804,396, U.S. Pat. No. 5,846,749, EP 711,565, WO 96/16673, U.S. Pat. No. 5,783,404, U.S. Pat. No. 5,977,322, U.S. Pat. No. 6,512,097, WO 97/00271, U.S. Pat. No. 6,270,765, U.S. Pat. No. 6,395,272, U.S. Pat. No. 5,837,243, WO 96/40789, U.S. Pat. No. 5,783,186, U.S. Pat. No. 6,458,356, WO 97/20858, WO 97/38731, U.S. Pat. No. 6,214,388, U.S. Pat. No. 5,925,519, WO 98/02463, U.S. Pat. No. 5,922,845, WO 98/18489, WO 98/33914, U.S. Pat. No. 5,994,071, WO 98/45479, U.S. Pat. No. 6,358,682 B1, US 2003/0059790, WO 99/55367, WO 01/20033, US 2002/0076695 A1, WO 00/78347, WO 01/09187, WO 01/21192, WO 01/32155, WO 01/53354, WO 01/56604, WO 01/76630, WO02/05791, WO 02/11677, U.S. Pat. No. 6,582,919, US2002/0192652A1, US 2003/0211530A1, WO 02/44413, US 2002/0142328, U.S. Pat. No. 6,602,670 B2, WO 02/45653, WO 02/055106, US 2003/0152572, US 2003/0165840, WO 02/087619, WO 03/006509, WO03/012072, WO 03/028638, US 2003/0068318, WO 03/041736, EP 1,357,132, US 2003/0202973, US 2004/0138160, U.S. Pat. No. 5,705,157, U.S. Pat. No. 6,123,939, EP 616,812 B1, US 2003/0103973, US 2003/0108545, U.S. Pat. No. 6,403,630 B1, WO 00/61145, WO 00/61185, U.S. Pat. No. 6,333,348 B1, WO 01/05425, WO 01/64246, US 2003/0022918, US 2002/0051785 A1, U.S. Pat. No. 6,767,541, WO 01/76586, US 2003/0144252, WO 01/87336, US 2002/0031515 A1, WO 01/87334, WO 02/05791, WO 02/09754, US 2003/0157097, US 2002/0076408, WO 02/055106, WO 02/070008, WO 02/089842 and WO 03/86467.

In order to develop treatment options for patients diagnosed with tumors that are non-responsive or respond poorly to treatment with a particular anti-cancer agent, such as a particular anti-HER2 antibody, there is a need for reliable robust cell lines and animal models that are suitable for evaluating various treatment modalities. In particular, there is a need for cell lines and animal models that enable the development of effective therapies for the treatment of HER2 positive cancer that is non-responsive or responds poorly to treatment with trastuzumab or other therapeutic agents, e.g. antibodies, that are similar to trastuzumab is their mechanism of action. Furthermore, there is a great need for cell lines and animal models for screening drug candidates for the treatment of ligand activated HER2 expressing tumors, including potential HER dimerization inhibitors (HDIs).

### SUMMARY OF THE INVENTION

In one aspect, the invention concerns a BT-474-based stable breast cancer cell line that (1) overexpresses HER2 at least at a 3+ level; (2) is non-reliant on estrogen supplementation for in vivo growth, and (2) does not respond or responds poorly to treatment with trastuzumab. In a specific embodiment, the cell line is the Exogenous Estrogen Independent breast cancer cell line designated BT-474EEI

In another embodiment the cell line is characterized by the fact that its growth is inhibited by a trastuzumab-cytotoxic agent conjugate, such as a trastuzumab-auristatin or a trastuzumab-DM1 conjugate.

In another embodiment, the cell line is immortalized.

In another aspect, the invention concerns a cell line as described above, which is obtained by multiple passages as xenografts in vivo and by intermittent in vitro culturing of a BT474 human mammary adenocarcinoma cell line, and by establishing a cell line from a transplanted tumor.

In another aspect, the invention concerns a model of HER2 overexpressing tumor that does not respond or responds poorly to treatment with trastuzumab, comprising the cell line described above.

The model can be a non-human animal model, where the animal may, for example, be a rodent, such as an immunocompromised rodent, e.g. a mouse or a rat.

In a further aspect, the invention concerns a method for identifying an agent for the treatment of a HER2 overexpressing tumor that does not respond or responds poorly to treatment with trastuzumab, comprising administering to a non-human animal carrying a BT-474-based tumor that: (1) overexpresses HER2 at least at a 3+ level; (2) is non-reliant on estrogen supplementation for in vivo growth, and (2) does not respond or responds poorly to treatment with trastuzumab, a candidate agent, and assessing tumor growth in the non-human animal, wherein inhibition of tumor growth compared to a control, non-treated non-human animal is indicative of the candidate being an agent for the treatment of HER2 overexpressing tumor. Just as before, the non-human animal can, for example, be a rodent, such as a mouse or a rat, e.g., an immunocompromised mouse or rat.

In a further aspect, the invention concerns a method of identifying an agent for increasing responsiveness to trastuzumab of a HER2 overexpressing tumor that does not respond or responds poorly to treatment with trastuzumab, comprising: administering to a non-human animal carrying a BT-474-based tumor that: (1) overexpresses HER2 at a 3+ level or above; (2) is non-reliant on estrogen supplementation for in vivo growth, and (3) does not respond or responds poorly to treatment with trastuzumab, a candidate agent in the presence of trastuzumab; and assessing tumor growth in said non-human animal, wherein inhibition of tumor growth compared to a control, non-treated non-human animal is indicative of the candidate being an agent for the treatment of HER2 overexpressing ligand activated tumor when used in combination with trastuzumab.

Any candidate agents can be screened by the method of the present invention, using the cell lines and animal models herein, including, without limitation, polypeptides, antibodies, antibody fragments, antibody-cytotoxic agent conjugates, and peptide and non-peptide small molecules.

The tumor preferably is breast cancer.

In another aspect, the invention concerns a method for identifying an agent for the treatment of HER2 overexpressing tumor that does not respond or responds poorly to treatment with trastuzumab, comprising contacting the culture of a cell line of the invention with a candidate agent, and assessing the growth of the cell line, wherein inhibition of growth compared to a control, is indicative of the candidate being an agent for the treatment of said HER2 overexpressing tumor.

In yet another aspect, the invention concerns a method for identifying an agent for the treatment of HER2 overexpressing tumor that does not respond or responds poorly to treatment with trastuzumab, comprising contacting the culture of a cell line with a candidate agent in the presence of trastuzumab and assessing the growth of said cell line, wherein inhibition of growth compared to a control is indicative of the candidate being an agent for the treatment of said HER2 overexpressing tumor when used in combination with trastuzumab.

The agents identified by the assays of the present invention can then be used alone or in combination with one or more other therapeutic agents, for example, trastuzumab to treat a patient diagnosed with a HER2 overexpressing tumor, such as breast cancer, not responding or responding poorly to treatment with trastuzumab.

### BRIEF DESCRIPTION OF THE DRAWINGS

FIG. 1: Growth of tumors from cells derived from BT-474JB tumors grown in the absence of estrogen supplementation. Cells were isolated from several BT-474JB tumors and put into cell culture. After several passages, the cells were harvested and inoculated into mice at the concentrations shown, with or without matrigel supplementation.

FIG. 2 shows results confirming the in vivo growth, without exogenous estrogen, of cell line BT-474JB, after passage in culture.

FIG. 3: Response of original BT-474JB tumors to naked HERCEPTIN® at different dose levels. BT-474JB cells form xenograft tumors in the presence of exogenous estrogen pellets. These xenograft tumors are characterized by high HER2 over-expression (3+) and are extremely sensitive to HERCEPTIN® treatment.

FIG. 4: BT-474EEI xenograft tumors treated with a single dose of naked HERCEPTIN® or HERCEPTIN®-Auristatin F Antibody Drug Conjugate (ADC).

FIG. 5: Effect of multiple doses of HERCEPTIN® or HERCEPTIN®-Auristatin F Antibody Drug Conjugate (ADC) and vehicle in Beige Nude Mice (20 million cells (in matrigel)/mouse).

FIG. 6: Extended Dose Response of HERCEPTIN®-DM1 Antibody Drug Conjugate (ADC) or HERCEPTIN® on BT-474EEI Xenograft Tumors in Beige Nude Mice (20 million cells (in matrigel)/mouse)

FIGS. 7A and B: Proliferation of BT-474 cells (FIG. 7A) and BT-474EEI (FIG. 7B) in response to naked trastuzumab and Trastuzumab-DM1 Antibody Drug Conjugate (ADC)

FIGS. 8A and B: Proliferation of BT-474 Cells (FIG. 8A) and BT-474EEI (8B) in response to Tarceva.

FIG. 9: HER2 expression on BT-474EEI cells, BT-474 cells, and BT474JB cells. P indicates passage number in culture.

FIG. 10: Western-blotting analysis of phosphoralation of HER receptors and downstream signalling molecules for BT-474EEI cells, BT-474 cells, and BT-474JB cells

FIGS. 11A and B: Total and phosphorylated AKT levels in BT-474EEI cells, BT-474 cells, and BT-474JB cells (FIG. 11A) as well as AKT kinase activity of BT-474EEI cells, BT-474 cells, and BT-474JB cells (FIG. 11B)

FIG. 12: MAP kinase (MAPK) activity of BT-474EEI cells, BT-474 cells, and BT-474JB cells

FIGS. 13A and B: Protein expression of IGF-1 receptor (IGF-1R) in BT-474EEI cells compared to BT-474 cells and BT-474JB cells both via Western-blotting analysis (FIG. 13A) and FACS analysis (FIG. 13B)

FIG. 13C: Microarray analysis of gene expression of IGF-1R in BT-474EEI cells compared to BT-474 cells and BT-474JB cells

FIG. 14: Effect of inhibition of activity of IGF-1R or silencing of IGF-1R gene expression on the responsiveness of BT-474EEI to Trastuzumab

FIG. 15: Gene expression of erbB receptors and ligands in BT-474EEI cells as compared to the BT-474 and BT-474JB cells measured by Taqman analysis

FIG. 16: Inhibition of BT-474 EEI cell growth by humanized anti-IGF-1R antibody hu10H5

FIG. 17: Synergistic inhibition of BT-474 EEI cell growth by antibody 10H5 and anti-estrogen ICI 82,780 (Faslodex)

### DETAILED DESCRIPTION OF THE PREFERRED EMBODIMENTS

### I. Definitions

A tumor which “does not respond, or responds poorly, to treatment with trastuzumab” does not show statistically significant improvement in response to trastuzumab treatment when compared to no treatment or treatment with placebo in a recognized animal model or a human clinical trial, or which responds to initial treatment with trastuzumab but grows as treatment is continued.

The terms “enhance responsiveness to trastuzumab” and “restore sensitivity to trastuzumab” are used interchangeably, and refer to a statistically significant improvement in response to trastuzumab treatment, so that a tumor which “does not respond, or responds poorly, to treatment with trastuzumab”, instead shows statistically significant improvement in response to trastuzumab treatment when compared to no treatment or treatment with trastuzumab alone in a recognized animal model or a human clinical trial. Such an enhancement preferably provides a partial response (PR), or more preferably, a completel response (CR) of a tumor to the treatment of trastuzumab.

The terms “responsiveness” and an “objective response” are used interchangeably, and refer to a measurable response, including complete response (CR) and partial response (PR).

By “complete response” or “CR” is intended the disappearance of all signs of cancer in response to treatment. This does not always mean the cancer has been cured.

“Partial response” or “PR” refers to a decrease of at least 50% in the size of one or more tumors or lesions, or in the extent of cancer in the body, in response to treatment.

A “HER receptor” is a receptor protein tyrosine kinase which belongs to the HER receptor family and includes EGFR, HER2, HER3 and HER4 receptors. The HER receptor will generally comprise an extracellular domain, which may bind an HER ligand and/or dimerize with another HER receptor molecule; a lipophilic transmembrane domain; a conserved intracellular tyrosine kinase domain; and a carboxyl-terminal signaling domain harboring several tyrosine residues which can be phosphorylated. The HER receptor may be a “native sequence” HER receptor or an “amino acid sequence variant” thereof. Preferably the HER receptor is native sequence human HER receptor.

The terms “ErbB1,” “HER1”, “epidermal growth factor receptor” and “EGFR” are used interchangeably herein and refer to EGFR as disclosed, for example, in Carpenter et al. Ann. Rev. Biochem. 56:881-914 (1987), including naturally occurring mutant forms thereof (e.g. a deletion mutant EGFR as in Humphrey et al. PNAS (USA) 87:4207-4211 (1990)). erbB 1 refers to the gene encoding the EGFR protein product.

The expressions “ErbB2” and “HER2” are used interchangeably herein and refer to human HER2 protein described, for example, in Semba et al., PNAS (USA) 82:6497-6501 (1985) and Yamamoto et al. Nature 319:230-234 (1986) (Genebank accession number X03363). The term “erbB2” refers to the gene encoding human ErbB2 and neu refers to the gene encoding rat p185^{neu} Preferred HER2 is native sequence human HER2.

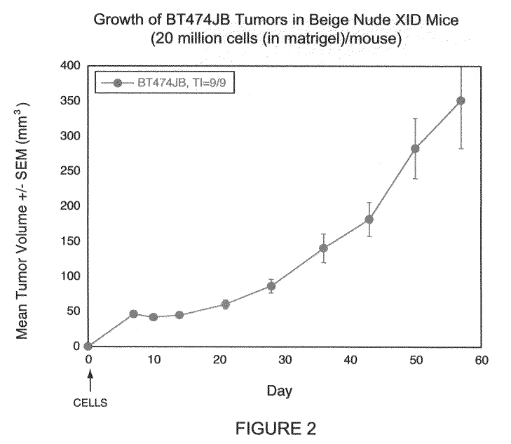

Herein, “HER2 extracellular domain” or “HER2ECD” refers to a domain of HER2 that is outside of a cell, either anchored to a cell membrane, or in circulation, including fragments thereof. In one embodiment, the extracellular domain of HER2 may comprise four domains: “Domain I” (amino acid residues from about 1-195;), “Domain II” (amino acid residues from about 196-319), “Domain III” (amino acid residues from about 320-488), and “Domain IV” (amino acid residues from about 489-630) (residue numbering without signal peptide). See Garrett et al. Mol. Cell. 11: 495-505 (2003), Cho et al. Nature 421: 756-760 (2003), Franklin et al. Cancer Cell 5:317-328 (2004), and Plowman et al. Proc. Natl. Acad. Sci. 90:1746-1750 (1993), as well as FIG. 2 herein.

“ErbB3” and “HER3” refer to the receptor polypeptide as disclosed, for example, in U.S. Pat. Nos. 5,183,884 and 5,480,968 as well as Kraus et al. PNAS (USA) 86:9193-9197 (1989).

The terms “ErbB4” and “HER4” herein refer to the receptor polypeptide as disclosed, for example, in EP Pat Appln No 599,274; Plowman et al., Proc. Natl. Acad. Sci. USA, 90:1746-1750 (1993); and Plowman et al., Nature, 366:473-475 (1993), including isoforms thereof, e.g., as disclosed in WO99/19488, published Apr. 22, 1999.

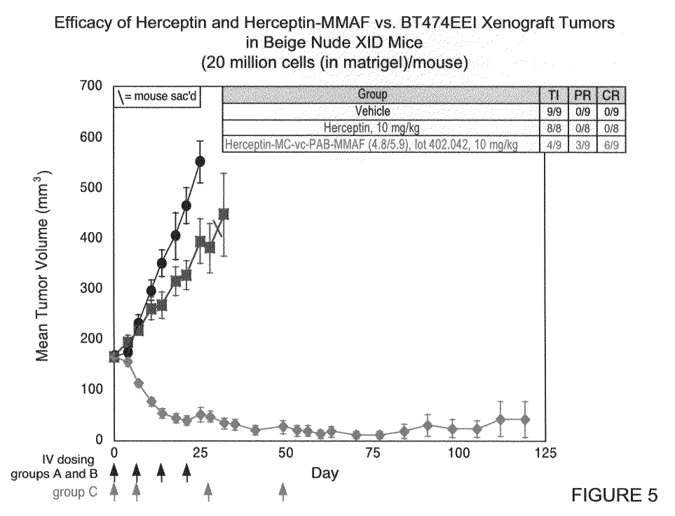

A “HER inhibitor” is an agent which interferes with HER activation or function. Examples of HER inhibitors include HER antibodies (e.g., EGFR, HER2, HER3, or HER4 antibodies); EGFR-targeted drugs; small molecule HER antagonists; HER tyrosine kinase inhibitors; HER2 and EGFR dual tyrosine kinase inhibitors such as lapatinib/GW572016; antisense molecules (see, for example, WO2004/87207); and/or agents that bind to, or interfere with function of, downstream signaling molecules, such as MAPK or Akt (see FIG. 5). Preferably, the HER inhibitor is an antibody or small molecule which binds to a HER receptor.

Protein “expression” refers to conversion of the information encoded in a gene into messenger RNA (mRNA) and then to the protein.

Herein, a sample or cell that “expresses” a protein of interest (such as a HER receptor or HER ligand) is one in which mRNA encoding the protein, or the protein, including fragments thereof, is determined to be present in the sample or cell.

The terms “progeny” and “progeny of the transgenic animal” refer to any and all offspring of every generation subsequent to the originally transformed mammals. The term “non-human mammal” refers to all members of the class Mammalia except humans. “Mammal” refers to any animal classified as a mammal, including humans, domestic and farm animals, and zoo, sports, or pet animals, such as mouse, rat, rabbit, pig, sheep, goat, cattle and higher primates.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced also increases in the proportion of the number of copies made of the particular gene expressed.

A “native sequence” polypeptide is one which has the same amino acid sequence as a polypeptide (e.g., HER receptor or HER ligand) derived from nature, including naturally occurring or allelic variants. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); U.S. Pat. No. 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995)).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

Humanized HER2 antibodies include huMAb4D5-1, huMAb4D5-2, huMAb4D5-3, huMAb4D5-4, huMAb4D5-5, huMAb4D5-6, huMAb4D5-7 and huMAb4D5-8 or trastuzumab as described in Table 3 of U.S. Pat. No. 5,821,337 expressly incorporated herein by reference; humanized 520C9 (WO93/21319); and humanized 2C4 antibodies such as pertuzumab as described herein.

For the purposes herein, “trastuzumab,” “HERCEPTIN®,” and “huMAb4D5-8” refer to an antibody comprising the light and heavy chain amino acid sequences in U.S. Pat. No. 5,821,337.

An “intact antibody” herein is one which comprises two antigen binding regions, and an Fc region. Preferably, the intact antibody has a functional Fe region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies; single-chain antibody molecules; and multispecific antibodies formed from antibody fragment(s).

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab=fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g. an antibody variable domain) and can be assessed using various assays as herein disclosed, for example.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes.” There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII. FcR expression on hematopoietic cells in summarized is Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g., from blood or PBMCs as described herein.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)), and regulates homeostasis of immunoglobulins.

“Complement dependent cytotoxicity” or “CDC” refers to the ability of a molecule to lyse a target in the presence of complement. The complement activation pathway is initiated by the binding of the first component of the complement system (C lq) to a molecule (e.g. an antibody) complexed with a cognate antigen. To assess complement activation, a CDC assay, e.g. as described in Gazzano-Santoro et al., J. Immunol. Methods 202:163 (1996), may be performed.

“Single-chain Fv” or “scFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. For a review of scFv see Plückthun in The Pharmacology of Monoclonal Antibodies, vol. 113, Rosenburg and Moore eds., Springer-Verlag, New York, pp. 269-315 (1994). HER2 antibody scFv fragments are described in WO93/16185; U.S. Pat. No. 5,571,894; and U.S. Pat. No. 5,587,458.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “naked antibody” is an antibody that is not conjugated to a heterologous molecule, such as a cytotoxic moiety or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by VH and VL domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

The term “main species antibody” herein refers to the antibody structure in a composition which is the quantitatively predominant antibody molecule in the composition.

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g., deamidated antibody variant), a basic variant, an antibody with an amino-terminal leader extension (e.g. VHS-) on one or two light chains thereof, an antibody with a C-terminal lysine residue on one or two heavy chains thereof, etc, and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieties attached thereto which differ from one or more carbohydrate moieties attached to a main species antibody. Examples of glycosylation variants herein include antibody with a G1 or G2 oligosaccharide structure, instead a G0 oligosaccharide structure, attached to an Fc region thereof, antibody with one or two carbohydrate moieties attached to one or two light chains thereof, antibody with no carbohydrate attached to one or two heavy chains of the antibody, etc, and combinations of glycosylation alterations.

Where the antibody has an Fc region, an oligosaccharide structure may be attached to one or two heavy chains of the antibody, e.g. at residue 299 (298, Eu numbering of residues).

Unless indicated otherwise, a “G1 oligosaccharide structure” herein includes G-1, G1-1, G1(1-6) and G1(1-3) structures.

An “amino-terminal leader extension” herein refers to one or more amino acid residues of the amino-terminal leader sequence that are present at the amino-terminus of any one or more heavy or light chains of an antibody. An exemplary amino-terminal leader extension comprises or consists of three amino acid residues, VHS, present on one or both light chains of an antibody variant.

A “deamidated” antibody is one in which one or more asparagine residues thereof has been derivitized, e.g., to an aspartic acid, a succinimide, or an iso-aspartic acid.

“Tumor”, as used herein, refers to all neoplastic cell growth and proliferation, whether malignant or benign, and all pre-cancerous and cancerous cells and tissues.

The terms “cancer” and “cancerous” refer to or describe the physiological condition in mammals that is typically characterized by unregulated cell growth. Examples of cancer include, but are not limited to, carcinoma, lymphoma, blastoma (including medulloblastoma and retinoblastoma), sarcoma (including liposarcoma and synovial cell sarcoma), neuroendocrine tumors (including carcinoid tumors, gastrinoma, and islet cell cancer), mesothelioma, schwannoma (including acoustic neuroma), meningioma, adenocarcinoma, melanoma, and leukemia or lymphoid malignancies. More particular examples of such cancers include squamous cell cancer (e.g. epithelial squamous cell cancer), lung cancer including small-cell lung cancer (SCLC), non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer (including metastatic breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

An “advanced” cancer is one which has spread outside the site or organ of origin, either by local invasion or metastasis.

A “refractory” cancer is one which progresses even though an anti-tumor agent, such as a chemotherapeutic agent, is being administered to the cancer patient. An example of a refractory cancer is one which is platinum refractory.

A “recurrent” cancer is one which has regrown, either at the initial site or at a distant site, after a response to initial therapy.

Herein, a “patient” is a human patient. The patient may be a “cancer patient,” i.e. one who is suffering or at risk for suffering from one or more symptoms of cancer.

A “tumor sample” herein is a sample derived from, or comprising tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

A “fixed” tumor sample is one which has been histologically preserved using a fixative.

A “formalin-fixed” tumor sample is one which has been preserved using formaldehyde as the fixative.

An “embedded” tumor sample is one surrounded by a firm and generally hard medium such as paraffin, wax, celloidin, or a resin. Embedding makes possible the cutting of thin sections for microscopic examination or for generation of tissue microarrays (TMAs).

A “paraffin-embedded” tumor sample is one surrounded by a purified mixture of solid hydrocarbons derived from petroleum.

Herein, a “frozen” tumor sample refers to a tumor sample which is, or has been, frozen.

A cancer or biological sample which “displays HER expression, amplification, or activation” is one which, in a diagnostic test, expresses (including overexpresses) a HER receptor, has amplified HER gene, and/or otherwise demonstrates activation or phosphorylation of a HER receptor.

A cancer or biological sample which “displays HER activation” is one which, in a diagnostic test, demonstrates activation or phosphorylation of a HER receptor. Such activation can be determined directly (e.g., by measuring HER phosphorylation by ELISA) or indirectly (e.g., by gene expression profiling or by detecting HER heterodimers, as described herein).

Herein, “gene expression profiling” refers to an evaluation of expression of one or more genes as a surrogate for determining HER phosphorylation directly.

A “phospho-ELISA assay” herein is an assay in which phosphorylation of one or more HER receptors, especially HER2, is evaluated in an enzyme-linked immunosorbent assay (ELISA) using a reagent, usually an antibody, to detect phosphorylated HER receptor, substrate, or downstream signaling molecule. Preferably, an antibody which detects phosphorylated HER2 is used. The assay may be performed on cell lysates, preferably from fresh or frozen biological samples.

A cancer cell with “HER receptor overexpression or amplification” is one which has significantly higher levels of a HER receptor protein or gene compared to a noncancerous cell of the same tissue type. Such overexpression may be caused by gene amplification or by increased transcription or translation. HER receptor overexpression may be determined in a diagnostic or prognostic assay by evaluating increased levels of the HER protein present on the surface of a cell (e.g., via an immunohistochemistry assay; IHC on tumors or via FACS on cells). Alternatively, or additionally, one may measure levels of HER-encoding nucleic acid in the cell, e.g., via fluorescent in situ hybridization (FISH; see WO98/45479 published October, 1998) or southern blotting. One may also study HER receptor overexpression by measuring shed antigen (e.g., HER extracellular domain) in a biological fluid such as serum (see, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al. J. Immunol. Methods 132: 73-80 (1990)). Aside from the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the antibody.

A cancer which “does not overexpress or amplify HER receptor” is one which does not have higher than normal levels of HER receptor protein or gene compared to a noncancerous cell of the same tissue type.

HER2 overexpression may be analyzed by IHC, e.g., using the HERCEPTEST® (Dako). Parrafin embedded tissue sections from a tumor biopsy may be subjected to the IHC assay and accorded a ErbB2 protein staining intensity criteria as follows:

Score 0 no staining is observed or membrane staining is observed in less than 10% of tumor cells.
Score 1+ a faint/barely perceptible membrane staining is detected in more than 10% of the tumor cells. The cells are only stained in part of their membrane.
Score 2+ a weak to moderate complete membrane staining is observed in more than 10% of the tumor cells.
Score 3+ a moderate to strong complete membrane staining is observed in more than 10% of the tumor cells.

Those tumors with scores of 0 for HER2 overexpression assessment may be characterized as not overexpressing HER2, whereas those tumors with 1+ scores may be characterized as having low overexpression of HER2, those tumors with 2+ scores may be characterized as moderately overexpressing HER2, and those tumors with 3+ scores may be characterized as highly overexpressing HER2.

Alternatively, or additionally, FISH assays such as the INFORM™ (sold by Ventana, Ariz.) or PATHVISION™ (Vysis, Ill.) may be carried out on formalin-fixed, paraffin-embedded tumor tissue to determine the extent (if any) of ErbB2 gene amplification in the tumor.

Herein, an “anti-tumor agent” refers to a drug used to treat cancer. Non-limiting examples of anti-tumor agents herein include chemotherapeutic agents, HER dimerization inhibitors, HER antibodies, antibodies directed against tumor associated antigens, anti-hormonal compounds, cytokines, EGFR-targeted drugs, anti-angiogenic agents, tyrosine kinase inhibitors, growth inhibitory agents and antibodies, cytotoxic agents, antibodies that induce apoptosis, COX inhibitors, farnesyl transferase inhibitors, antibodies that binds oncofetal protein CA 125, HER2 vaccines, Raf or ras inhibitors, liposomal doxorubicin, topotecan, taxane, dual tyrosine kinase inhibitors, TLK286, EMD-7200, pertuzumab, trastuzumab, erlotinib, and bevacizumab.

An “approved anti-tumor agent” is a drug used to treat cancer which has been accorded marketing approval by a regulatory authority such as the Food and Drug Administration (FDA) or foreign equivalent thereof.

By “standard of care” herein is intended the anti-tumor agent or agents that are routinely used to treat a particular form of cancer. For example, for platinum-resistant ovarian cancer, the standard of care is topotecan or liposomal doxorubicin.

A “growth inhibitory agent” when used herein refers to a compound or composition which inhibits growth of a cell, especially a HER expressing cancer cell either in vitro or in vivo. Thus, the growth inhibitory agent may be one which significantly reduces the percentage of HER expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine) and taxanes. Those agents that arrest G1 also spill over into S-phase arrest, for example, DNA alkylating agents such as dacarbazine, mechlorethamine and topo II inhibitors such as doxorubicin, epirubicin, daunorubicin, and etoposide. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13.

Examples of “growth inhibitory” antibodies are those which bind to HER2 and inhibit the growth of cancer cells overexpressing HER2. Preferred growth inhibitory HER2 antibodies inhibit growth of SK-BR-3 breast tumor cells in cell culture by greater than 20%, and preferably greater than 50% (e.g. from about 50% to about 100%) at an antibody concentration of about 0.5 to 30 μg/ml, where the growth inhibition is determined six days after exposure of the SK-BR-3 cells to the antibody (see U.S. Pat. No. 5,677,171 issued Oct. 14, 1997). The SK-BR-3 cell growth inhibition assay is described in more detail in that patent and hereinbelow. The preferred growth inhibitory antibody is a humanized variant of murine monoclonal antibody 4D5, e.g., trastuzumab.

An antibody which “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies). The cell is usually one which overexpresses the HER2 receptor. Preferably the cell is a tumor cell, e.g. a breast, ovarian, stomach, endometrial, salivary gland, lung, kidney, colon, thyroid, pancreatic or bladder cell. In vitro, the cell may be a SK-BR-3, BT474, Calu 3 cell, MDA-MB-453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody which induces apoptosis is one which results in about 2 to 50 fold, preferably about 5 to 50 fold, and most preferably about 10 to 50 fold, induction of annexin binding relative to untreated cell in an annexin binding assay using BT474 cells (see below). Examples of HER2 antibodies that induce apoptosis are 7C2 and 7F3.

The “epitope 4D5” is the region in the extracellular domain of HER2 to which the antibody 4D5 (ATCC CRL 10463) and trastuzumab bind. This epitope is close to the transmembrane domain of HER2, and within Domain IV of HER2. To screen for antibodies which bind to the 4D5 epitope, a routine cross-blocking assay such as that described in Antibodies, A Laboratory Manual, Cold Spring Harbor Laboratory, Ed Harlow and David Lane (1988), can be performed. Alternatively, epitope mapping can be performed to assess whether the antibody binds to the 4D5 epitope of HER2 (e.g. any one or more residues in the region from about residue 529 to about residue 625, inclusive of the HER2 ECD, residue numbering including signal peptide).

“Treatment” refers to both therapeutic treatment and prophylactic or preventative measures. Those in need of treatment include those already with cancer as well as those in which cancer is to be prevented. Hence, the patient to be treated herein may have been diagnosed as having cancer or may be predisposed or susceptible to cancer.

The term “effective amount” refers to an amount of a drug effective to treat cancer in the patient. The effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete respose, CR), increase overall survival time, and/or improve one or more symptoms of cancer (e.g. as assessed by FOSI).

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; TLK 286 (TELCYTAJ); acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; bisphosphonates, such as clodronate; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)) and anthracyclines such as annamycin, AD 32, alcarubicin, daunorubicin, dexrazoxane, DX-52-1, epirubicin, GPX-100, idarubicin, KRN5500, menogaril, dynemicin, including dynemicin A, an esperamicin, neocarzinostatin chromophore and related chromoprotein enediyne antiobiotic chromophores, aclacinomysins, actinomycin, authramycin, azaserine, bleomycins, cactinomycin, carabicin, caminomycin, carzinophilin, chromomycinis, dactinomycin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin, liposomal doxorubicin, and deoxydoxorubicin), esorubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; folic acid analogues such as denopterin, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, and floxuridine; androgens such as calusterone, dromostanolone propionate, epitiostanol, mepitiostane, and testolactone; anti-adrenals such as aminoglutethimide, mitotane, and trilostane; folic acid replenisher such as folinic acid (leucovorin); aceglatone; anti-folate anti-neoplastic agents such as ALIMTA®, LY231514 pemetrexed, dihydrofolate reductase inhibitors such as methotrexate, anti-metabolites such as 5-fluorouracil (5-FU) and its prodrugs such as UFT, S-1 and capecitabine, and thymidylate synthase inhibitors and glycinamide ribonucleotide formyltransferase inhibitors such as raltitrexed (TOMUDEX™, TDX); inhibitors of dihydropyrimidine dehydrogenase such as eniluracil; aldophosphamide glycoside; aminolevulinic acid; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK7 polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); cyclophosphamide; thiotepa; taxoids and taxanes, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; gemcitabine (GEMZAR®); 6-thioguanine; mercaptopurine; platinum; platinum analogs or platinum-based analogs such as cisplatin, oxaliplatin and carboplatin; vinblastine (VELBAN®); etoposide (VP-16); ifosfamide; mitoxantrone; vincristine (ONCOVIN®); vinca alkaloid; vinorelbine (NAVELBINE®); novantrone; edatrexate; daunomycin; aminopterin; xeloda; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovorin.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

An “antimetabolite chemotherapeutic agent” is an agent which is structurally similar to a metabolite, but can not be used by the body in a productive manner. Many antimetabolite chemotherapeutic agents interfere with the production of the nucleic acids, RNA and DNA. Examples of antimetabolite chemotherapeutic agents include gemcitabine (GEMZAR®), 5-fluorouracil (5-FU), capecitabine (XELODA®), 6-mercaptopurine, methotrexate, 6-thioguanine, pemetrexed, raltitrexed, arabinosylcytosine ARA-C cytarabine (CYTOSAR-U®), dacarbazine (DTIC-DOME®), azocytosine, deoxycytosine, pyridmidene, fludarabine (FLUDARA®), cladrabine, 2-deoxy-D-glucose etc. The preferred antimetabolite chemotherapeutic agent is gemcitabine.

“Gemcitabine” or “2′-deoxy-2′,2′-difluorocytidine monohydrochloride (b-isomer)” is a nucleoside analogue that exhibits antitumor activity. The empirical formula for gemcitabine HCl is C9H11F2N3O4 A HCl. Gemcitabine HCl is sold by Eli Lilly under the trademark GEMZAR®.

A “platinum-based chemotherapeutic agent” comprises an organic compound which contains platinum as an integral part of the molecule. Examples of platinum-based chemotherapeutic agents include carboplatin, cisplatin, and oxaliplatinum.

By “platinum-based chemotherapy” is intended therapy with one or more platinum-based chemotherapeutic agents, optionally in combination with one or more other chemotherapeutic agents.

By “chemotherapy-resistant” cancer is meant that the cancer patient has progressed while receiving a chemotherapy regimen (i.e. the patient is “chemotherapy refractory”), or the patient has progressed within 12 months (for instance, within 6 months) after completing a chemotherapy regimen.

By “platinum-resistant” cancer is meant that the cancer patient has progressed while receiving platinum-based chemotherapy (i.e. the patient is Aplatinum refractory@), or the patient has progressed within 12 months (for instance, within 6 months) after completing a platinum-based chemotherapy regimen.

An “anti-angiogenic agent” refers to a compound which blocks, or interferes with to some degree, the development of blood vessels. The anti-angiogenic factor may, for instance, be a small molecule or antibody that binds to a growth factor or growth factor receptor involved in promoting angiogenesis. The preferred anti-angiogenic factor herein is an antibody that binds to vascular endothelial growth factor (VEGF), such as bevacizumab (AVASTIN®).

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ, colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

As used herein, the term “EGFR-targeted drug” refers to a therapeutic agent that binds to EGFR and, optionally, inhibits EGFR activation. Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX7) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF (see WO98/50433, Abgenix); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding; and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). Examples of small molecules that bind to EGFR include ZD1839 or Gefitinib (IRESSA®; Astra Zeneca); CP-358774 or Erlotinib (TARCEVA®; Genentech/OSI); and AG1478, AG1571 (SU 5271; Sugen); EMD-7200.

A “tyrosine kinase inhibitor” is a molecule which inhibits tyrosine kinase activity of a tyrosine kinase such as a HER receptor. Examples of such inhibitors include the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; GW572016 (available from Glaxo) an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibits Raf-1 signaling; non-HER targeted TK inhibitors such as Imatinib mesylate (Gleevac®) available from Glaxo; MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino)phthalimide); tyrphostines containing nitrothiophene moieties; PD-0183805 (Wamer-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); Inatinib mesylate (Gleevac; Novartis); PKI 166 (Novartis); GW2016 (Glaxo SmithKline); CI-1033 (Pfizer); EKB-569 (Wyeth); Semaxinib (Sugen); ZD6474 (AstraZeneca); PTK-787 (Novartis/Schering AG); INC-1C11 (Imclone); or as described in any of the following patent publications: U.S. Pat. No. 5,804,396; WO99/09016 (American Cyanimid); WO98/43960 (American Cyanamid); WO97/38983 (Warner Lambert); WO99/06378 (Warner Lambert); WO99/06396 (Warner Lambert); WO96/30347 (Pfizer, Inc); WO96/33978 (Zeneca); WO96/3397 (Zeneca); and WO96/33980 (Zeneca).

A “fixed” or “flat” dose of a therapeutic agent herein refers to a dose that is administered to a human patient without regard for the weight (WT) or body surface area (BSA) of the patient. The fixed or flat dose is therefore not provided as a mg/kg dose or a mg/m² dose, but rather as an absolute amount of the therapeutic agent.

A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks.

Antibodies with improved binding to the neonatal Fc receptor (FcRn), and increased half-lives, are described in WO00/42072 (Presta, L.) and US2005/0014934A1 (Hinton et al.). These antibodies comprise an Fc region with one or more substitutions therein which improve binding of the Fc region to FcRn. For example, the Fc region may have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising antibody variant with improved FcRn binding comprises amino acid substitutions at one, two or three of positions 307, 380 and 434 of the Fc region thereof (Eu numbering of residues).

The phrase “in the presence of trastuzumab” and “used in combination with trastuzumab” are used interchangeably and refer to the administration of the agent, identified by the assay of the present invention, and trastuzumab to a culture medium of a cell line or to a non-human animal or to a patient at an effective amount. The agent can be added or administered prior to, simutanously with, or subsequently to trastuzumab. Preferably, the patient is a cancer patient.

### II. Detailed Description

In HER2 positive tumor cells, the HER2 receptor tyrosine kinase can be activated by various mechanisms, including overexpression and ligand-mediated activation of another HER receptor.

Thus, HER2 overexpression/amplification is known to play a key role in tumorigenesis and cancer metastasis, including various adenocarcinomas, hormone refractory prostate cancer, and certain gastric, endometrial, ovarian, colon, and lung cancers. In particular, HER2 overexpression has been recognized to play a central role in the tumorigenesis and metastasis of certain breast adenocarcinomas. In breast cancer, genomic amplification and overexpression of HER2 is predictive of poor prognosis (Slamon et al., Science 235:177-182 (1987). Breast cancer patients whose tumors overexpress HER2 are candidates for treatment with trastuzumab (Herceptin®, Genentech, Inc.).

The present invention concerns HER2 overexpressing tumor cell lines that are not reliant on exogenous estrogen supplementation for in vivo growth and do not respond or respond poorly to treatment with trastuzumab. The novel cell lines of the invention were developed from a cell line designated BT-474JB. BT-474JB (obtained from Jose Baselga, Vall d'Hebron University Hospital. Barcelona, Spain) is a derivative of BT-474, a HER2 expressing breast carcinoma cell line (ATCC HTB 20), which is known to show an antiproliferative response to mAb 4D5 (trastuzumab) treatment. See, e.g., Motoyama et al., Cancer Res 62:3151-3158 (2002).

Thus, the present invention is based on experimental results obtained with a transplant line developed from a variant of the commercially available BT-474 human mammary adenocarcinoma cell line (ATCC HTB 20), designated BT-474JB. Through multiple passages as xenografts in vivo and intermittent in vitro culturing of the human mammary adenocarcinoma BT-474JB cell line, a new cell line, designated BT-474EEI has been developed, that is non-reliant on exogenous estrogen supplementation for in vivo growth and is a high HER2 (at least about 3+) expresser. This cell line forms xenograft tumors that express high levels of HER2 and are not responsive or respond poorly to treatment with trastuzumab. This is particularly interesting as the cell line from which it is derived is exquisitely sensitive to trastuzumab treatment. Accordingly, the BT-474JB-based cell line and animal models are useful to test treatment options for the treatment of high HER2 expressing tumors that do not respond or respond poorly to treatment with trastuzumab.

Thus, the present invention provides useful cell lines and animal models for evaluating new therapies targeting HER2 overexpressing tumors that show poor or no responsiveness to treatment with trastuzumab. In a particular embodiment, the tumor-bearing animals and cell lines are useful in screening compounds that have potential as prophylactic or therapeutic treatments of ligand-activated HER2 expressing tumors. In another embodiment, the tumor bearing animals and cell lines of the present invention are useful to test trastuzumab-drug conjugates for prophylactic or therapeutic treatments of tumors that do not respond or respond poorly for treatment with trastuzumab alone. In yet another embodiment, the tumor-bearing animals and cell lines are useful in screening compounds that act as enhancers for enhancing tumor cells' responsiveness to trastuzumab, and therefore, have potential as prophylactic or therapeutic treatments of ligand-activated HER2 expressing tumors when used in combination with trastuzumab. In this embodiment, even if a candidate compound alone does not have sufficient potential as prophylactic or therapeutic treatments of ligand-activated HER2 expressing tumors, it may enhance the sensitivity (responsiveness) of these tumors to trastuzumab. Therefore, the combination of the candidate agent and trastuzumab is capable of inhibiting tumor growth as compared to a control.

Screening for a useful drug involves administering the candidate drug over a range of doses to the tumor-bearing animal, and assaying at various time points for the effect(s) of the drug on the disease or disorder being evaluated. Alternatively, or additionally, the drug can be administered prior to or simultaneously with exposure to an inducer of the disease, if applicable.

Tumor bearing animals include all non-human mammals, such as, for example, higher primates, domestic and farm animals, rodents, such as mouse, rat, guinea pig, and zoo, sports, or pet animals, such as rabbit, pig, sheep, goat, cattle. Preferred recipient animals are rodents, in particular mice and rats.

### Screening Assays

In one embodiment, candidate compounds are screened by being administered to the tumor-bearing animal over a range of doses, and evaluating the animal's physiological response to the compounds over time. Administration may be oral, or by suitable injection, depending on the chemical nature of the compound being evaluated. In some cases, it may be appropriate to administer the compound in conjunction with co-factors that would enhance the efficacy of the compound.

If cell lines are used to screen for compounds useful in treating various disorders associated with HER2-overexpression, the test compounds are added to the cell culture medium at an appropriate time, and the cellular response to the compound is evaluated over time using the appropriate biochemical and/or histological assays. In some cases, it may be appropriate to apply the compound of interest to the culture medium in conjunction with co-factors that would enhance the efficacy of the compound.

Thus, the present invention provides assays for identifying agents which are antagonists of the abnormal cellular function of the overexpressed HER2 protein in the pathogenesis of cellular proliferation and/or differentiation of mammary gland that is causally related to the development of breast tumors, in particular breast tumors the pathogenesis of which involves ligand-mediated HER2 activation. Similarly, the present invention provides assays for identifying agents interfering with the abnormal cellular function of overexpressed HER2 protein in other HER2 overexpressing cancers, including, without limitation, ovarian and non-small cell lung cancers.

In addition to screening a drug for use in treating a disease or condition, the animals of the present invention are also useful in designing a therapeutic regimen aimed at preventing or curing the disease or condition. For example, the animal may be treated with a combination of a particular diet, exercise routine, radiation treatment, chemotherapy and/or one or more compounds identified herein either prior to, simultaneously, or after the onset of the disease or condition. Such an overall therapy or regimen might be more effective at combating the disease or condition than treatment with a compound alone.

Agents to be tested in the animals and cell cultures of the present invention can be produced, for example, by bacteria, yeast or other organisms (e.g., natural products), produced chemically (e.g., small molecules, including peptidomimetics), or by techniques of recombinant DNA technology or gene activation (e.g., polypeptides, including antibodies and antibody fragments).

One may assess the growth inhibitory effects of a test compound on the cell lines of the present invention, including cell lines derived from the tumor-bearing animals herein, e.g., essentially as described in Schaefer et al. Oncogene 15: 1385-1394 (1997). According to this assay, the cells may be treated with a test compound at various concentrations for 4 days and stained with crystal violet. Incubation with the compound may show a growth inhibitory effect on this cell line similar to that displayed by monoclonal antibody 2C4 on MDA-MB-175-VII cells (Schaefer et al., supra). In a further embodiment, exogenous HRG will not significantly reverse this inhibition.

To identify growth inhibitory compounds that specifically target HER2, one may screen for compounds which inhibit the growth of HER2-overexpressing cancer cells. To identify such compounds, the assay described in U.S. Pat. No. 5,677,171 can be performed. According to this assay, HER2 overexpressing cells are grown in a 1:1 mixture of F12 and DMEM medium supplemented with 10% fetal bovine serum, glutamine and penicillin streptomycin. The cells are plated at 20,000 cells in a 35 mm cell culture dish (2 mls/35 mm dish) and the test compound is added at various concentrations. After six days, the number of cells, compared to untreated cells is counted using an electronic COULTER™ cell counter. Those compounds which inhibit cell growth by about 20-100% or about 50-100% may be selected as growth inhibitory compounds.

The above-described approaches can also be used to select for compounds which enhance the responsiveness of tumors to trastuzumab. The trastuzumab can be administered at various dosages, prior to, subsequent to, or simultaneously with the administration of the candicate compounds to the HER2 overexpressing cells. Those compounds which inhibit cell growth by about 20-100% or about 50-100% in the presence of trastuzumab may be selected as trastuzumab-responsiveness enhancer compounds

To select for compounds which induce cell death, loss of membrane integrity as indicated by, e.g., PI, trypan blue or 7AAD uptake may be assessed relative to control. The preferred assay is the PI uptake assay using cells isolated from the breast tumor tissue of the tumor-bearing animal. According to this assay, the cells are cultured in Dulbecco's Modified Eagle Medium (D-MEM):Ham's F-12 (50:50) supplemented with 10% heat-inactivated FBS (Hyclone) and 2 mM L-glutamine. (Thus, the assay is performed in the absence of complement and immune effector cells). The cells are seeded at a density of 3×10⁶ per dish in 100×20 mm dishes and allowed to attach overnight. The medium is then removed and replaced with fresh medium alone or medium containing various concentrations of the compound. The cells are incubated for a 3-day time period. Following each treatment, monolayers are washed with PBS and detached by trypsinization. Cells are then centrifuged at 1200 rpm for 5 minutes at 4.degree. C., the pellet resuspended in 3 ml ice cold Ca²⁺ binding buffer (10 mM Hepes, pH 7.4, 140 mM NaCl, 2.5 mM CaCl.sub.2) and aliquoted into 35 mm strainer-capped 12.times.75 tubes (1 ml per tube, 3 tubes per treatment group) for removal of cell clumps. Tubes then receive PI (10 μg/ml). Samples may be analyzed using a FACSCAN™ flow cytometer and FACSCONVERT™ CellQuest software (Becton Dickinson). Those compounds which induce statistically significant levels of cell death as determined by PI uptake may be selected as cell death-inducing compounds.

In order to select for compounds which induce apoptosis, an annexin binding assay using cells established from the breast tumor tissue of the transgenic animal is performed. The cells are cultured and seeded in dishes as discussed in the preceding paragraph. The medium is then removed and replaced with fresh medium alone or medium containing 10 μg/ml of the monoclonal antibody. Following a three-day incubation period, monolayers are washed with PBS and detached by trypsinization. Cells are then centrifuged, resuspended in Ca.sup.2+binding buffer and aliquoted into tubes as discussed above for the cell death assay. Tubes then receive labeled annexin (e.g. annexin V-FTIC) (1.μg/ml). Samples may be analyzed using a FACSCANT™ flow cytometer and FACSCONVERT™. CellQuest software (Becton Dickinson). Those compounds which induce statistically significant levels of annexin binding relative to control are selected as apoptosis-inducing compounds.

Candidate Molecules for Screening according to the Invention

The candidate molecules screened using the cell lines and animal models of the present invention include polypeptides, including antibodies and antibody fragments, peptide and non-peptide small molecules, and the like.

In a particular embodiment, the cell lines and animal models herein are used to screen antibodies for the treatment of HER2 overexpressing tumors that do not respond or respond poorly to treatment with trastuzumab. Such tumors preferably show strong HER2 expression (grade 3+ or above typically).

In another embodiment, the cell lines and animal model are useful in screening polypeptides or antibodies that act as enhancers for improving tumor cells' responsiveness to trastuzumab. Such polypeptides or antibodies preferably show increased or decreased expression or activity in the cells lines of the present invention as compared to the parental control.

In another embodiment, the cell lines and animal models of the present invention can be used to test the efficacy of antibody conjugates for the prevention and/or treatment of HER2 overexpressing tumors that do not respond or respond poorly to treatment with trastuzumab. Such conjugates include conjugates of an antibody and one or more small molecule cytotoxic drugs, such as a calicheamicin, a maytansine (U.S. Pat. No. 5,208,020), a trichothene, or CC1065. In one embodiment of the invention, the candidate compound screened is an anti-HER2 antibody conjugated to one or more maytansine molecules (e.g., about 1 to about 10 maytansine molecules per antibody molecule). Maytansinoids inhibit cell proliferation by inhibiting tubulin polymerization. Maytansine may, for example, be converted to May-SS-Me, which may be reduced to May-SH3 and reacted with modified antibody (Chari et al. Cancer Research 52: 127-131 (1992)) to generate a maytansinoid-antibody immunoconjugate.

In a specific embodiment, the antibody conjugate is a trastuzumab-MC-vc-PAB-MMAF conjugate, wherein MC-vc-PAB is a linker and stands for Maleiamide Caproyl-Val-Cit-paraminobenzoic acid (the PAB self immolates and is released). MMAF stands for MonoMethyl-Auristatin F (a derivative of MonoMethyl-Auristatin E with a phenylalanine at the C-terminus of the drug), a known anticancer drug.

### III. Deposit of Materials

The following hybridoma cell lines have been deposited with the American Type Culture Collection, 10801 University Boulevard, Manassas, Va. 20110-2209, USA (ATCC):

| Antibody Designation | ATCC No. | Deposit Date |
|---|---|---|
| 7C2 | ATCC HB-12215 | Oct. 17, 1996 |
| 7F3 | ATCC HB-12216 | Oct. 17, 1996 |
| 4D5 | ATCC CRL 10463 | May 24, 1990 |
| 2C4 | ATCC HB-12697 | Apr. 8, 1999 |

The foregoing written specification is considered to be sufficient to enable one skilled in the art to practice the invention. The present invention is not to be limited in scope by the constructs deposited, since the deposited embodiments are intended to illustrate only certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims. Indeed, various modifications of the invention in addition to those shown and described herein will become apparent to those skilled in the art from the foregoing description and fall within the scope of the appended claims.

It is understood that the application of the teachings of the present invention to a specific problem or situation will be within the capabilities of one having ordinary skill in the art in light of the teachings contained herein.

Further details of the invention are illustrated by the following non-limiting Examples. The disclosures of all citations in the specification are expressly incorporated herein by reference.

### Example 1

### Establishment of Exogenous Estrogen Independent Tumor Cell Line BT-474EEI

BT-474JB Cells

The BT-474JB cells were obtained from Jose Baselga (Vall d'Hebron University Hospital, Barcelona, Spain.). The JB cells were derived by obtaining a BT-474 xenograft tumor and re-establishing the cells in culture. 10 million BT-474 cells, which do not grow well as xenografts, were implanted subcutaneously into female BALB/c nude mice, which had been implanted the day before with 0.72 mg estrogen pellets. A fast-growing tumor was obtained to put back into culture. These cells can then be grown up for further xenograft studies.

Preparation of BT-474EEI Cells

The BT-474JB cells were grown in DMEM:Ham's F-12 medium containing 10% FBS and 2 mM 1-glutamine. Ten week old female beige nude mice (Harlan Sprague Dawley, Madison, Wis.) were implanted subcutaneously with 0.36 mg estrogen pellets (Innovative Research of America, Sarasota, Fla.) 1-2 days prior to being inoculated with 20 million BT-474JB cells from the above cell culture into the mammary fat pad. One of the tumors that developed from this inoculation was transplanted into the mammary fat pads of 5 beige nude mice (without estrogen supplement, transplant 1). All future passages were without estrogen supplement.

From transplant 1, a mouse with a tumor showing strong to moderate HER2 staining by immunohistochemistry was used as a donor for the next transplant (transplant 2).

Similarly, from transplant 2, a mouse whose tumor showed strong to moderate homogenous HER2 staining was selected for the next transplant (transplant 3). Interestingly, the HER2 positive cells of this tumor were later shown by histology to be surrounded by hematolymphoid cells of murine origin.

From transplant 3, two mice were selected as donors for the next transplantation (transplant 4). Histologically the tumors from these mice did stain for HER2, and had characteristics of a hematolymphoid neoplasm.

From transplant 4, 8 of the 10 tumors generated were collected, minced, and introduced back into cell culture. As the hematolymphoid tumor cells were not adherent in culture, they were easily removed leaving behind the HER2 positive tumor cells originated from the BT-474JB cell line.

Cells were again passaged in culture and were again inoculated into female beige nude mice. Tumors grew well and those from the matrigel-supplemented groups were macerated and reintroduced into cell culture.

These cells were expanded and inoculated into female beige nude mice. It was confirmed that these cells did indeed grow well in vivo and immunohistochemistry indicated that this cell line was HER2 3+. This line was at this point dubbed BT-474EEI (BT474 Exogenous Estrogen Independent). The EEI (Exogenous Estrogen Independent) designation was given, as this is the first BT-474 derived line that does not need exogenous estrogen supplementation for in vivo growth.

### Example 2

### BT-474EEI Xenograft Trastuzumab Efficacy Studies

Cells were grown in DMEM: Ham's F-12 medium containing 10% FBS and 2 mM 1-glutamine. They were detached from the flasks with 5 mM EDTA in 150 mM NaCl, washed and resuspended in culture medium. The cell suspension is mixed 50:50 with cold phenol red-free matrigel (Becton Dickinson) for a final cell number of 10⁸ per ml. Mice were injected with 0.2 ml for 20 million cells per mouse inoculum.

Female Beige nude mice (Harlan Sprague Dawley, Madison, Wis.) 6-8 weeks of age were injected with 2×10⁷ BT-474EEI cells into the mammary fat pad in a volume of 0.2 ml. Tumors were allowed to grow and were then measured in two dimensions using a caliper. Tumor volume was expressed in mm³ using the formula: V=(a×b²)×0.5, where a and b are the long and the short diameters of the tumor, respectively. Tumors were measured and mice were grouped into groups of 9 mice with a mean tumor volume between 100-200 mm 3. Trastuzumab at 10 mg/kg and a Vehicle Control were injected IV once a week for 4 weeks (Day 0, 7, 14, 24). Trastuzumab-MC-vc-PAB-MMAF antibody drug conjugate was injected on days 0, 7, 26 and 49. In the trastuzumab-MC-vc-PAB-MMA Fantibody conjugate, MC-vc-PAB is a linker, which stands for Maleiamide Caproyl, val-cit, paraminobenzoic acid (the PAB self immolates and is released). MMAF is the cytotoxic agent MonoMethyl-Auristatin F (a derivative of MonoMethyl-Auristatin E with a phenylalanine at the C-terminus). Tumors were measured twice a week throughout the experiment. Mice were sacrificed before mean tumor volumes reached 1000 mm3. All animal protocols were approved by an Institutional Use and Care Committee.

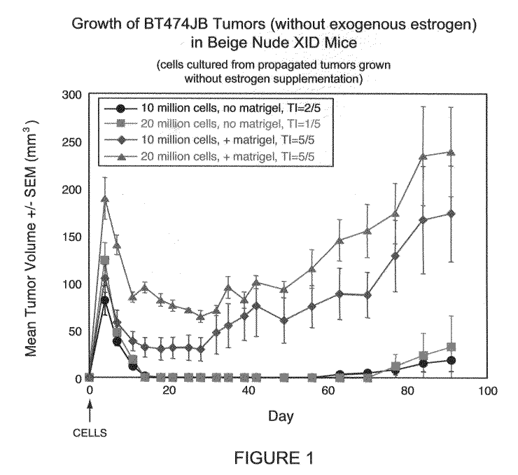

The results are shown in FIGS. 1-3.

FIG. 1 shows the growth of tumors from cells derived from BT-474JB tumors grown without estrogen supplementation. Cells were isolated from several BT474JB tumors and put into cell culture. After several passages, the cells were harvested and inoculated into mice at the concentrations shown, with or without matrigel supplementation.

FIG. 2 confirms the in vivo growth, without exogenous estrogen, of the new line, BT-474EEI, after passage in culture.

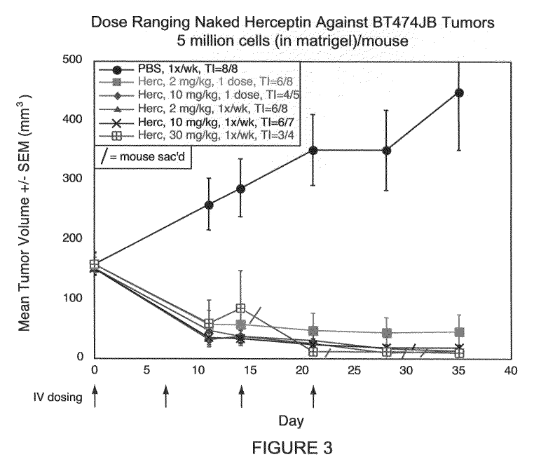

FIG. 3 shows the response of original BT-474JB tumors to naked trastuzumab at different dose levels. BT-474JB cells form xenograft tumors in the presence of exogenous estrogen pellets. These xenograft tumors are high HER2 expressers (3+) and are extremely sensitive to trastuzumab treatment.

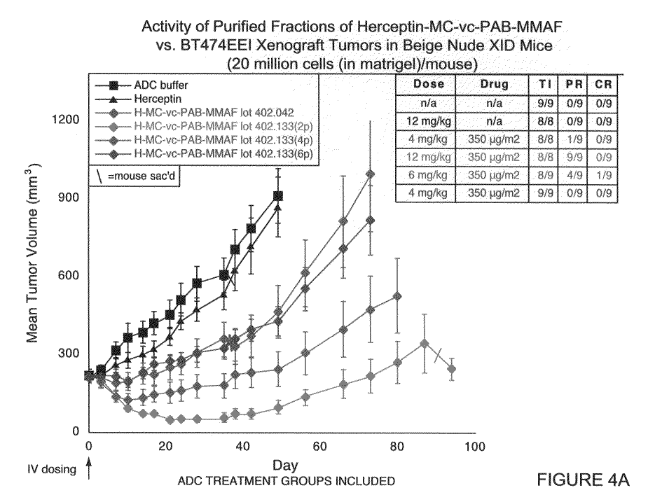

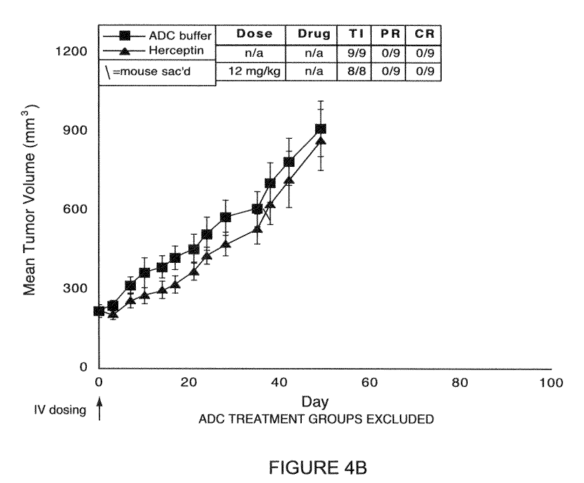

FIG. 4 shows the growth of BT-474EEI xenograft tumors treated with naked trastuzumab and a trastuzumab antibody drug conjugate (ADC). BT-474EEI cells grow well in vivo without exogenous estrogen supplementation and by immunohistochemistry (IHC) are strong HER2 expressers (3+). This HER23+line is substantially less sensitive to trastuzumab treatment compared to the BT-474JB line from which it was derived. However, upon treatment with an armed trastuzumab-MC-vc-PAB-MMAF conjugate that targets the cytotoxic (microtubule polymerization inhibitor) drug, MMAF, to HER2 positive cells, these xenografts are significantly inhibited.

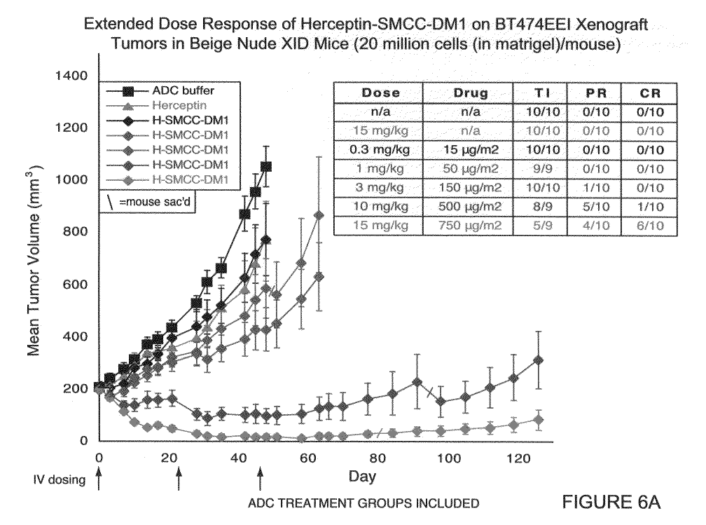

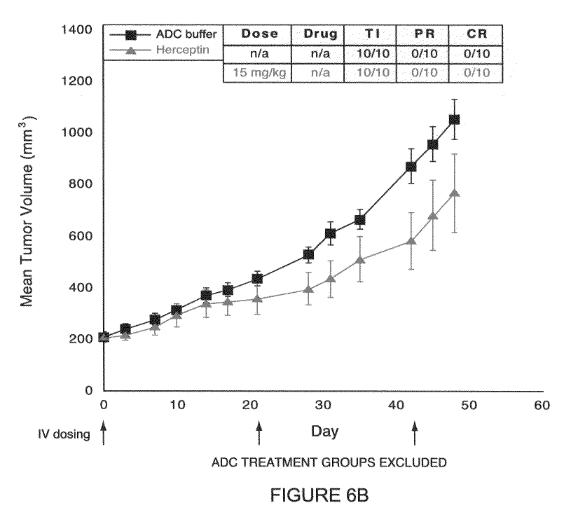

The in vivo results shown in FIGS. 5 and 6 further confirm that BT-474EEI xenograft tumors are substantially less sensitive to treatment with trastuzumab (HERCEPTIN®) than to treatment with an armed trastuzumab-MC-vc-PAB-MMAF conjugate that targets the cytotoxic (microtubule polymerization inhibitor) drug, MMAF, to HER2 positive cells. The difference in sensitivity is apparent at various doses.

As illustrated by the foregoing results, through multiple passages as xenografts in vivo and intermittent in vitro culturing of the human mammary adenocarcinoma BT-474JB cell line, a new cell line, dubbed BT-474EEI, was developed that is non-reliant on exogenous estrogen supplementation for in vivo growth and is a high HER2 (3+) expresser. This cell line may be passaged in culture and directly injected into the mammary fat pad of female beige nude mice to form xenograft tumors. Though this line forms tumors that express high levels of HER2, it is significantly less sensitive (potentially insensitive) to treatment with trastuzumab. This is particularly interesting as the cell line from which it is derived is exquisitely sensitive to trasuzumab treatment. Though naked trastuzumab does not show activity vs. BT-474EEI xenograft tumors, the armed antibody conjugate, trastuzumab-MC-vc-PAB-MMAF shows significant anti-tumor inhibition. Based on these results, the BT-474EEI cell line and the corresponding xenografts and tumor-bearing animals offer a useful model for investigating therapies targeting high HER2 expressing, trastuzumab-resistant/insensitive tumors.

### Example 3

### In Vitro Cell Proliferation Assay

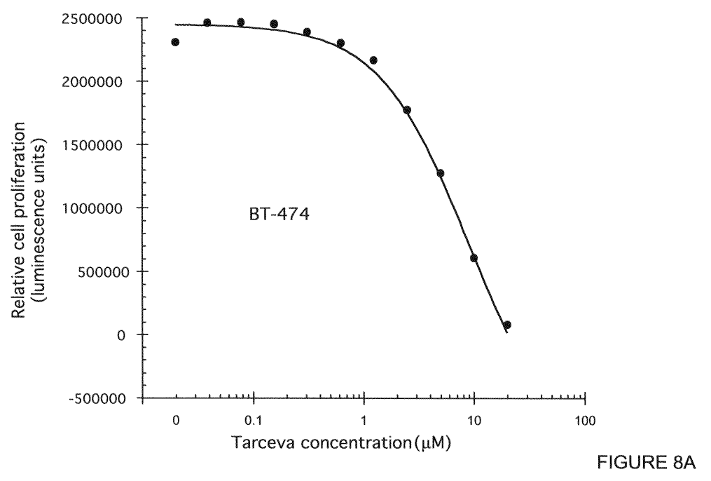

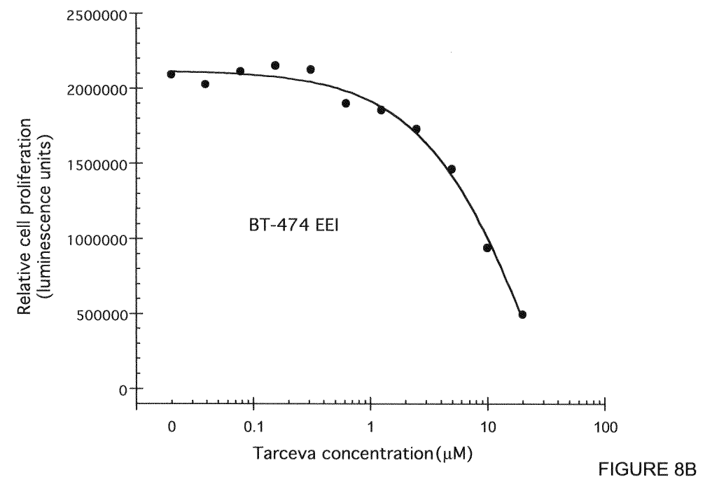

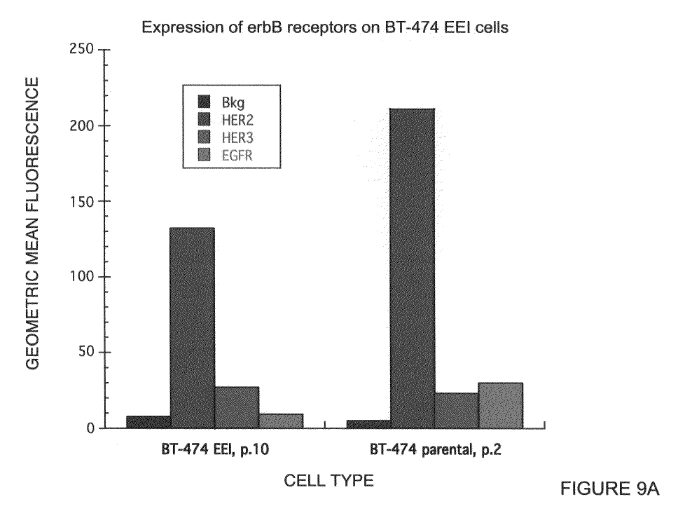

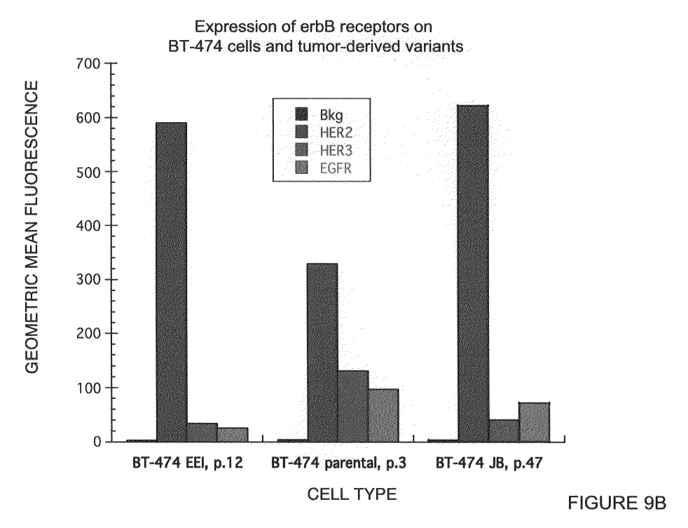

BT-474 and BT-474EE1 cells were plated in 96-well microtiter plates and incubated overnight at 37° in a humidified atmosphere of 5% CO2. Cells were treated with a range of concentrations of trastuzumab, trastuzumab-MCC-DM1, or Tarceva from 0.0001 ug/ml to 10 ug/ml. After incubation for 72 h, Cell Titer-Glo reagent (Promega, Madison, Wis.) was added to the wells, and after a 10-min incubation at room temperature, the luminescent signal was recorded. The results were shown in FIGS. 8-9.

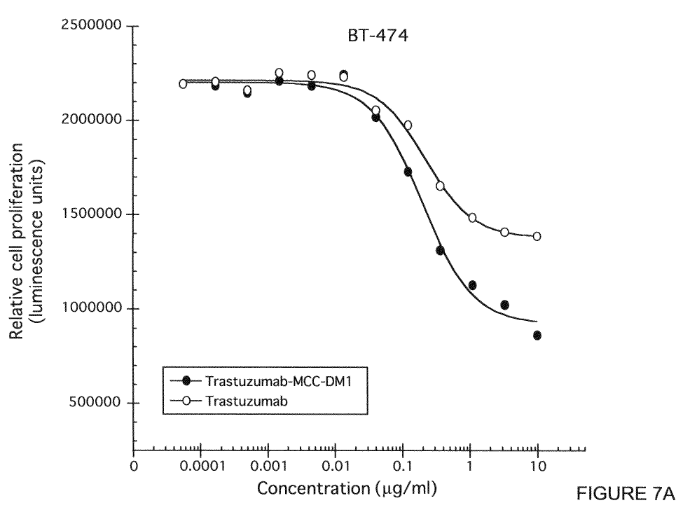

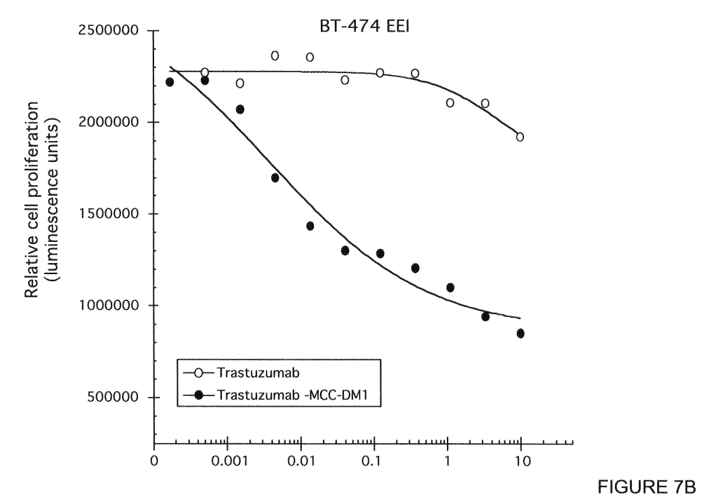

FIG. 7A shows the proliferation of BT-474 cells in response to naked trastuzumab and a trastuzumab antibody drug conjugate (ADC) at different dose levels. Cell proliferation was substantially reduced in a dose-dependent manner upon treatment with naked trastuzumab and trastuzumab antibody drug conjugate (ADC) (Trastuzumab-MCC-DM1), suggesting that BT-474 cells are very sensitive to both naked trastuzumab treatment and armed trastuzumab.

FIG. 7B shows the proliferation of BT-474EEI cells treated with naked trastuzumab and a trastuzumab antibody drug conjugate (ADC). Cell proliferation of BT-474EEI substantially decreased in a dose-dependent manner upon treatment with trastuzumab-antibody drug conjugate (ADC) (Trastuzumab-MCC-DM1), but had little change in response to naked trastuzumab, suggesting that BT474EEI cells are very sensitive only to armed trastuzumab, but not to naked trastuzumab.

FIG. 8A shows the proliferation of BT-474 cells in response to treatment with Tarceva at different dose levels. BT-474 cell proliferation decreased with high doses of Tarceva.

FIG. 8B shows the proliferation of BT-474EEI cells treated with Tarceva. Cell proliferation of the BT-474EEI cells was reduced at high concentrations of Tarceva.

The in vitro results shown in FIG. 7 further confirm that BT474EEI cells are substantially less sensitive to treatment with naked trastuzumab (HERCEPTIN®) than to treatment with an armed trastuzumab-MC-vc-PAB-MMAF conjugate that targets the cytotoxic (microtubule polymerization inhibitor) drug, MMAF, to HER2 positive cells. The difference in sensitivity is apparent at various doses.

### Example 4

### Protein Expression Study of BT474EEI Cells

In order to further uncover the molecular mechanism of the non-responsiveness to trastuzumab, protein expression of the HER receptors and molecules related to HER2 signaling pathway were examined using standard western blotting and FACS analysis.

Western Blotting—Cells were lysed in lysis buffer. Lysates were cleared by centrifugation and protein was quantitated using the BCA Protein Assay Kit (Pierce, Rockford, Ill.). Proteins were resolved by SDS-PAGE, transferred to nitrocellulose, and immunoblotted with the indicated primary antibodies, followed by incubation with horseradish peroxidase-conjugated secondary antibodies (Amersham Biosciences, Piscataway, N.J.) and visualization using enhanced chemiluminescence reagents (Amersham Biosciences, Piscataway, N.J.).

FACS Analysis: Cells were detached with 5 mM EDTA/150 mM NaCl, resuspended in 1% FBS/PBS (FACS buffer) and incubated for 2 hours at 4 degrees C. with 10 ug/ml anti-HER2 MAb 4D5, anti-HER3 MAb 2F9 or anti-EGFR MAb 6C5 or no primary antibody (background or Bkg). Cells were then washed and resuspended in buffer containing 15 ug/ml FITC-conjugated goat anti-mouse antibody and incubated for 1 hour at 4 degrees C. Cells were then washed once, resuspended in 1 ml buffer and analyzed on a Becton Dickinson FACScan.

FACS analysis revealed that BT-474EEI cells retain a high level of protein expression of HER2 (FIG. 9).

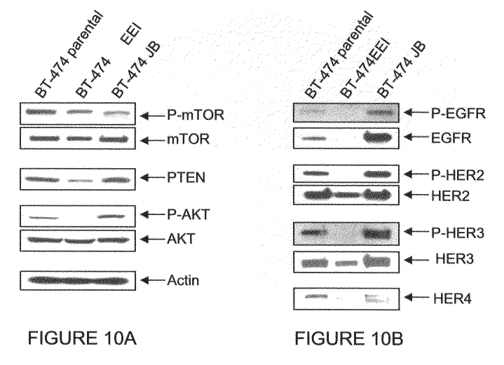

Western-blotting analysis further revealed that BT-474EEI cells do not have activated (constitutively phosphorylated) HER receptors. As shown in FIGS. 10A and B, while Western-blotting analysis detected in parental cells BT-474 and BT-474JB positive signals of phosphorylated EGF receptor (P-EGFR), phosphorylated HER2 (P-HER2), phosphorylated HER3 (P-HER3), phosphorylated AKT (P-AKT), no positive signal of these phosphorylated molecules was detected in BT-474EEI cells.

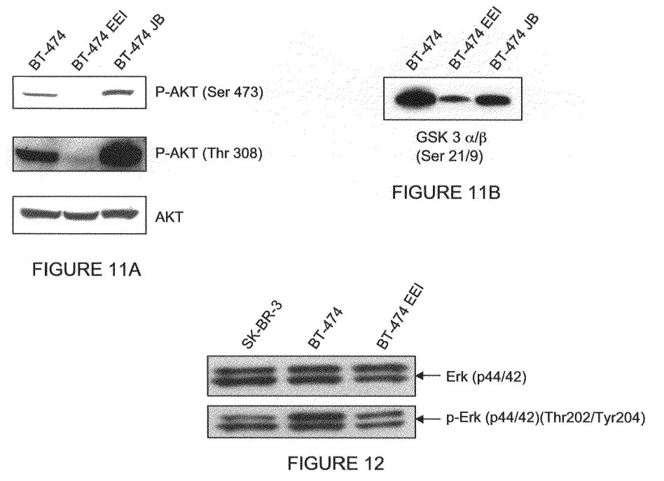

FIGS. 11A and B further confirm the results of FIGS. 10 A and B. FIG. 11A shows that, while total AKT level of BT-474EEI is comparable with that of the parental cells BT-474 and BT-474JB, the phosphorylated AKT level of BT-474EEI cells is less than that of BT-474 cells and BT-474JB cells. FIG. 11B validates the results of FIG. 11A by showing that BT-474EEI cells display reduced AKT kinase activity compared to the BT-474 cells and BT-474JB cells.

In contrast to AKT kinase, MAP kinase (MAPK) was shown to be activated in BT-474 EEI cells. FIG. 12 shows that BT-474EEI cells have the same amount of total Erk and phosphorylated Erk (p-Erk) as BT-474 cells and BT-474JB cells.

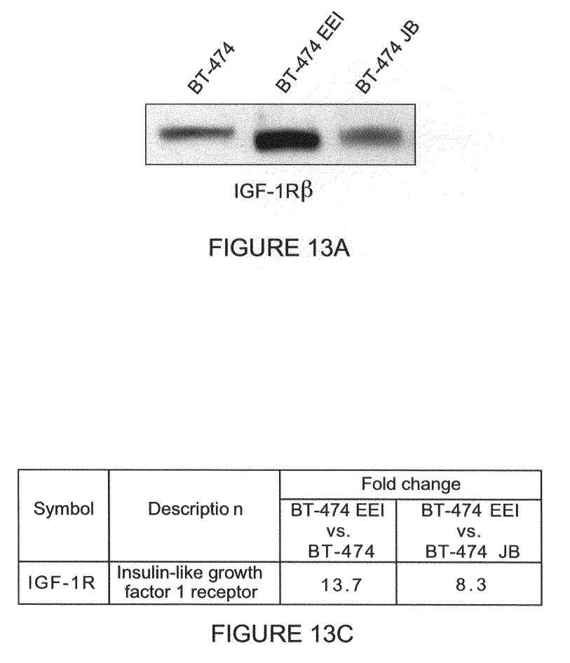

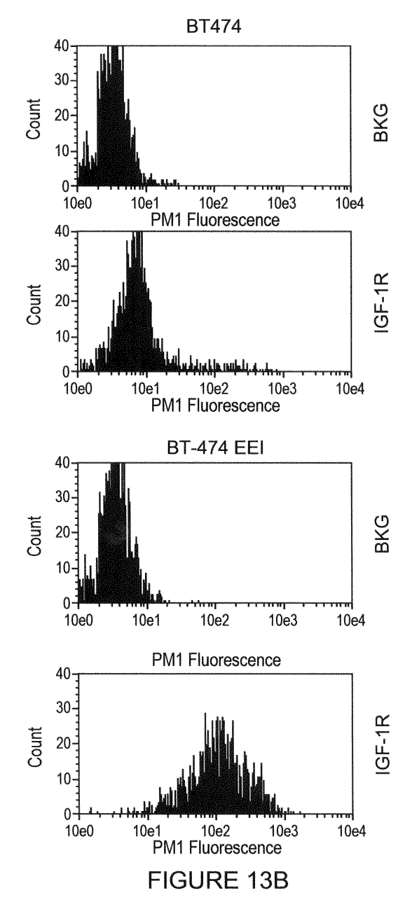

Expression of the IGF-1 receptor was also examined in the BT474EEI cells. FIG. 13 shows that the expression of IGF-1 receptor (IGF-1R) was higher in BT-474EEI compared to BT-474 cells and BT-474JB cells. This was shown by western blotting analysis (FIG. 13A) and FACS analysis (FIG. 13B).

FIG. 13C shows the expression of the IGF-1R gene with microarray analysis. Total RNA was labeled using standard Affymetrix labeling protocol that incorporates biotin labeled nucleotides into the cRNA. cRNA were hybridized to Affymetrix HGU133Av2 arrays. The arrays were washed and stained with streptavidin-pycoerythrin and scanned using the Affymetrix GeneArray scanner. Consistent with the protein expression data, microarray analysis (FIG. 13C) shows that mRNA expression of IGF-1R in the BT-474 EEI cells increases by about 8.3 fold as compared to the BT-474 JB cells and 13.7 fold as compared to the BT-474 cells

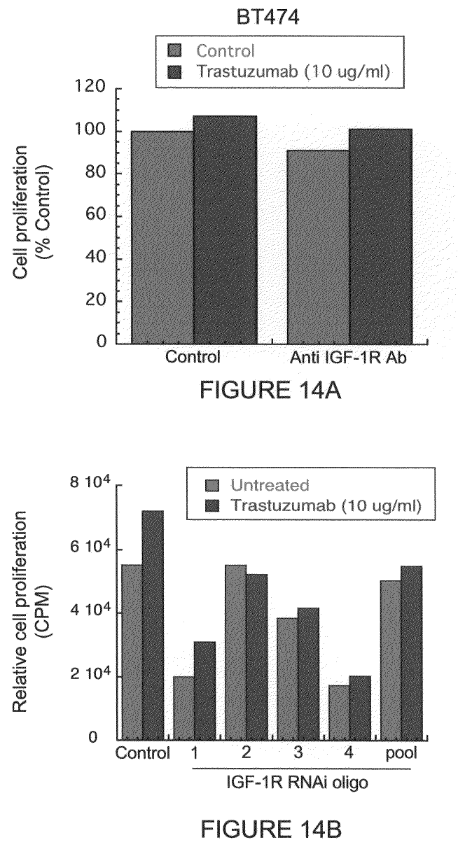

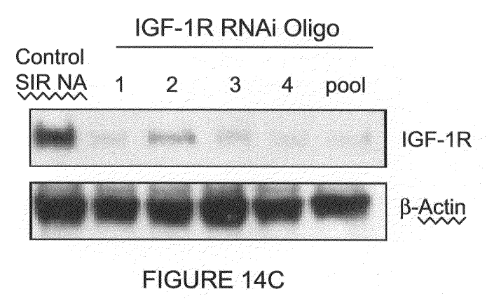

FIG. 14 shows that inhibition of IGF-1R activity or the silencing of IGF-1R gene expression had no effect on the responsiveness of BT-474EEI to trastuzumab. The activity of IGF-1R was inhibited with a neutralizing anti-IGF-1R antibody and the effect of such inhibition on the responsiveness to trastuzumab of BT-474EEI was examined. FIG. 14A shows that inhibition of IGF-1R activity does not restore sensitivity of BT-474EEI to trastuzumab. The mRNA encoding IGF-1R was also silenced with IGF-1R RNAi oligonucleotides. FIG. 14B indicates that silencing of the IGF-1R gene does not restore sensitivity of BT-474EEI to trastuzumab either.

### Example 5

### Gene Expression Profile of the BT-474EEI Cells

### Taqman Analysis

The gene expression profile of the BT-474EEI cells was further examined with Real Time Quantitative PCR (TaqMan) Analysis as described previously (Heid C. A. et al., Genome Res 6:986-994 (1996)). The sequences of the primer/probe sets used for this analysis are as follows:

EGFR:

(SEQ ID NO:1)

F 5′-GCCTTGAGTCATCTATTCAAGCAC-3′, R

(SEQ ID NO:2)

5′-TGCTACTGTCATTCGCACCTG-3′,

(SEQ ID NO:3)

P 5′-FAM-AGCTCTGGCCACAACAGGGCATTTT-TAMRA-p-3′;
HER-2/neu:

(SEQ ID NO:4)

F 5′-TCTGGACGTGCCAGTGTGAA-3′,

(SEQ ID NO:5)

R 5′-TGCTCCCTGAGGACACATCA-3′,

(SEQ ID NO:6)

P 5′-FAM-CAGAAGGCCAAGTCCGCAGAAGCC-TAMRA-p-3′;
HER-3:

(SEQ ID NO:7)

F 5′-TTCTCTACTCTACCATTGCCCAAC-3′,

(SEQ ID NO:8)

R 5′-CACCACTATCTCAGCATCTCGGTC-3′,

(SEQ ID NO:9)

P 5′-FAM-ACACCAACTCCAGCCACGCTCTGC-TAMRA-p-3′;
HER-4:

(SEQ ID NO:10)

F 5′-GAGATAACCAGCATTGAGCACAAC-3′,

(SEQ ID NO:11)

R 5′-AGAGGCAGGTAACGAAACTGATTA-3′,

(SEQ ID NO:12)

P 5′-FAM CCTCTCCTTCCTGCGGTCTGTTCGA-TAMRA-p-3′;
EGF:

(SEQ ID NO:13)

F 5′-AGCTAACCCATTATGGCAACA-3′,

(SEQ ID NO:14)

R 5′-AGTTTTCACTGAGTCAGCTCCAT-3′,

(SEQ ID NO:15)

P 5-FAM-AGGGCCCTGGACCCACCAC-TAMRA-p-3′;
TGF-α:

(SEQ ID NO:16)

F 5′-GGACAGCACTGCCAGAGA-3′,

(SEQ ID NO:17)

R 5′-CAGGTGATTACAGGCCAAGTAG-3′,

(SEQ ID NO:18)

P 5′-FAM-CCTGGGTGTGCCACAGACCTTC-TAMRA-p-3′;
HRG:

(SEQ ID NO:19)

F 5′-TGGCTGACAGCAGGACTAAC-3′,

(SEQ ID NO:20)

R 5′-CTGGCCTGGATTTCTTC-3′,

(SEQ ID NO:21)

P 5′-FAM-CAGCAGGCCGCTTCTCGACAC-TAMRA-p-3′;
amphiregulin:

(SEQ ID NO:22)

F 5′-ATATCACATTGGAGTCACTGCCCA-3′,

(SEQ ID NO:23)

R 5′-GGGTCCATTGTCTTATGATCCAC-3′,

(SEQ ID NO:24)

P 5′-FAM-AGCCATAAATGATGAGTCGGTCCTCTTTCC-TAMRA-
p-3′;
HB-EGF:

(SEQ ID NO:25)

F 5′-GAAAGACTTCCATCTAGTCACAAAGA-3′,

(SEQ ID NO:26)

R 5′-GGGAGGCCCAATCCTAGA-3,

(SEQ ID NO:27)

P 5-FAM-TCCTTCGTCCCCAGTTGCCG-TAMRA-p-3′;
betacellulin,

(SEQ ID NO:28)

F 5′-TGCCCCAAGCAATACAAGC-3′,

(SEQ ID NO:29)

R 5′-CGTCTGCTCGGCCACC-3′,

(SEQ ID NO:30)

P 5′-FAM-AAGCGGCATCTCCCTTTGATGCAGTAA-TAMRA-p-3′;
Epiregulin:

(SEQ ID NO:31)

F 5′-TGCATGCAATTTAAAGTAACTTATTTGACTA-3′,

(SEQ ID NO:32)

R 5′-ATCTTAAGGTACACAATTATCAAAGCTGA-3′,

(SEQ ID NO:33)

P 5′-FAM-TCGGATTACTGAATTGTATCAATTTGTTTGTGTTCA-
TAMRA-p-3′,
Estrogen Receptor (ER):

(SEQ ID NO:34)

F 5′-AGACGGACCAAAGCCACTTG-3′

(SEQ ID NO:35)

R 5′-CCCCGTGATGTAATACTTTTGCA-3′

(SEQ ID NO:36)

P 5′-FAM-CCACTGCGGGCTCTACTTCATCGC-TAMRA-p-3′

F and R are the forward and reverse primers, respectively, and P is the TaqMan probe (FAM as reporter, TAMRA as quencher). Human cDNA FLJ22101 fis (GenBank™ accession number AK025754) was used as a housekeeping gene for normalization of EGFR family receptor and ligand gene expression. Primer/probe sets for FLJ22101 are as follows:

(SEQ ID NO:37)

F 5′-TTCCCTGTGGCACTTGACATT-3′,

(SEQ ID NO:38)

R 5′-CTTTTGCCTCTGGCAGTACTCA-3′,
and

(SEQ ID NO:39)

P 5′-FAM-TGTCTTAAAGTTTTTGAAGTACATCTTCTGGCCCC-
TAMRA-p-3′.

Taqman One-Step Universal Master Mix (Product # 4309169, from Applied Biosystems, Foster City, Calif.) was used for all reactions (Applied Biosystems (2005): Real-time PCR Systems, Chemistry Guide (Rev.E)). TaqMan reaction was performed in a standard 96-well plate format with ABI 7500 Real-Time qPCR System. 100 ng total RNA was used for each reaction. For data analysis, raw Ct was first normalized to a housekeeping gene for each sample to get dCt. The normalized dCt was then calibrated to BT474 control to get ddct. In the final step of data analysis, the ddct was converted to fold change (2^{-ddCt}) relative to control.

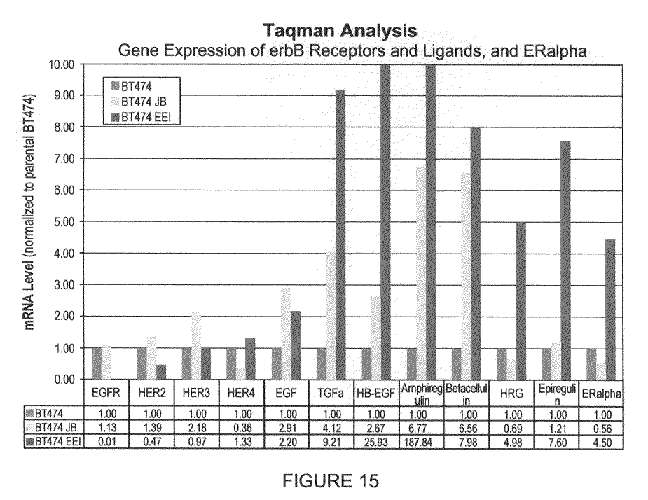

FIG. 15 shows gene expression of erbB receptors and ligands in BT-474EEI cells as compared to the BT-474 and BT-474JB cells measured by Taqman analysis. The gene expression results indicate that a number of genes encoding erbB receptors and ligands are differentially expressed in BT-474EEI cells as compared to the BT-474 and BT-474JB cells. For instance, gene expression of amphiregulin increased (more than 150 fold) in BT-474EEI cells as compared to the BT-474 and BT-474JB cells. In addition, betacellulin, HB-EGF and epiregulin are also significantly overexpressed in BT-474EEI cells. Such a differential gene expression profile is useful to uncover the molecular mechanism of the unresponsiveness of BT-474EEI to trastuzumab, and further identify modulating agents that decrease or eliminate such unresponsiveness.

### Example 6

### Gene Expression Profile of the BT-474EEI Cells

### Microarray Analysis

Nucleic acid microarrays, often containing thousands of gene sequences, are useful for identifying differentially expressed genes in diseased tissues as compared to their normal counterparts. Using nucleic acid microarrays, test and control mRNA samples from test and control samples are reverse transcribed and labeled to generate cDNA probes. The cDNA probes are then hybridized to an array of nucleic acids immobilized on a solid support. The array is configured such that the sequence and position of each member of the array is known. Hybridization of a labeled probe with a particular array member indicates that the sample from which the probe was derived expresses that gene. If the hybridization signal of a probe from a test (BT-474EEI cells) sample is greater than hybridization signal of a probe from a control (parental or JB cells) sample, the gene or genes overexpressed in the BT-474EEI cells are identified. The implication of this result is that an overexpressed protein in BT-474EEI cells is useful to study trastuzumab-resistance mechanism.

The methodology of hybridization of nucleic acids and microarray technology is well known in the art. In the present example, total RNA was extracted from triplicate flasks of BT-474, JB and EEI cells, then labeled using standard Affymetrix labeling protocol that incorporates biotin labeled nucleotides into the cRNA. cRNA were hybridized to Affymetrix HGU133Av2 arrays. The arrays were washed and stained with streptavidin-pycoerythrin and scanned using the Affymetrix GeneArray scanner. The results were summarized in Tables 1 and 2.

Table 1 sumarizes genes that are overexpressed significantly in BT474EEI cells as compared to BT-474 or BT-474JB cells and shows that increased expression is observed in genes involved in a variety of protein families, including ECM/cell surface molecules, cytokines, cell cycle/apoptosis molecules, ER signaling molecules, protein tyrosine kinase molecules, Ras/MAPK molecules, phosphatases, and proteases, etc.

Table 2 summarizes genes that have decreased expression in BT-474EEI cells as compared to BT-474 or BT-474JB cells.

TABLE 1

| | GENE OVEREXPRESSED | FOLD |
|---|---|---|
| GENE FAMILY | IN BT-474EE1 | OVEREXPRESSION |
| ECM/cell surface | Fibronectin | >299x |
| | Collagens IX, XIII | 30x |
| | Protocadherins | α, β, γ; 10-150x |
| | CD44 | 25-30x |
| | Mucin | 15 (80-170x) |
| | Galectin 1 | 70-1400x |
| Cytokines | IFN-inducible TMP1 | 150x |
| | IFN-inducible TMP2 | 20-30x |
| | IFN-inducible PK3 | 20-50x |
| | Fax | 20x |
| | regulator Fas-apoptosis | 10x |
| | JAK2 | 20x |
| Cell cycle/apoptosis | Cyclin A1 | 4-10x |
| | BCL-10 | 6-10x |
| | BNIPL | 5-9x |
| | p21 cip1 | 20x |
| ER signaling | ESR1 | 3-7x |
| | ERRRβ | 37x |
| | CBP/p300 | 10x |
| | C/EBP | 6x |
| | PS2 | 8-12x |
| | BCAS2 | 5x |
| | BCAS3 | 4-8x |
| | RERG | 5-12x |
| IGF-1 family | IGF-1R | 10-20x |
| | IRSI | 20-200x |
| | IGF-BP4 | 3-15x |
| | IGF-BP5 | 40x, EI/JB |
| | IGF-BP6 | 7x |
| EGFR ligands | Amphiregulin | 50-200x |
| | Betacellulin | 4-30x |
| | HB-EGF | 15x |
| | Epiregulin | 5x ½ probe sets |
| | Grb14 | 20x |
| | EGFR decreased* | |
| Ras/MAPK | N-ras | 4-15x |
| | Ras p21 activators 3, 4 | 5-20x |
| | MAPKKK5 | 3-XXx |
| | MAPKK5 | 30-40x |
| | MAPK6 | 4-5x |
| | Rab proteins (11, 27A, 31, | 30-400x |
| | 34, 41) | |
| | decreased ‘ras-induced | 0.1x |
| | senescence’ | |
| Phosphatases | PPF1A4 | 28x EI/JB |
| | PTPIE | 3-10x |
| | PTPLA | 1-10x |
| | PTPN22 | 0.2-6x |
| | DUSP 23 | 20x |
| Proteases/inhibitors | NMP1 | 9-15x |
| | NMP13 | 11x |
| | NMP 19 | 13x |
| | TIMP1 | 13-45x |
| | TIMP2 | 2-20x |
| | TIMP3 | 10-200x |
| | SERPINA3 | 170x |
| | SERPINA5 | 60-80x |
| | SERPINE1 | 5-10x |

TABLE 2
Decreased expression EEI vs. parental and JB

| | |
|---|---|
| Bad | SMO |
| Survivin | |
| Caspase 3 | EGFR |
| Cdc2 | |
| CDC25A | Grb 7 |
| | Topo IIA |
| Ras-induced senescence | |
| RhoGTPase activating protein | 17β-OH-steriod DH |

### Example 7

### Gene Amplification Analysis of the BT-474EEI Cells

Gene amplification or loss in BT-474EEI cells was determined with purified genomic DNA by SNP analysis using the standard Affymetrix procedures. A wide variety of techniques have been developed for SNP detection and analysis in the art, see, e.g. Sapolsky et al. (1999) U.S. Pat. No. 5,858,659; Shuber (1997) U.S. Pat. No. 5,633,134; Dahlberg (1998) U.S. Pat. No. 5,719,028; Murigneux (1998) WO98/30717; Shuber (1997) WO97/10366; Murphy et al. (1998) WO98/44157; Lander et al. (1998) WO98/20165; Goelet et al. (1995) WO95/12607 and Cronin et al. (1998) WO98/30883. In addition, ligase based methods are described by Barany et al. (1997) WO97/31256 and Chen et al. Genome Res. 1998; 8(5):549-56; mass-spectroscopy-based methods by Monforte (1998) WO98/12355, Turano et al. (1998) WO98/14616 and Ross et al. (1997) Anal Chem. 15, 4197-202; PCR-based methods by Hauser, et al. (1998) Plant J. 16, 117-25; exonuclease-based methods by Mundy U.S. Pat. No. 4,656,127; dideoxynucleotide-based methods by Cohen et al. WO91/02087; Genetic Bit Analysis or GBA™ by Goelet et al. WO92/15712; Oligonucleotide Ligation Assays or OLAs by Landegren et al. (1988) Science 241:1077-1080 and Nickerson et al. (1990) Proc. Natl. Acad. Sci. (U.S.A.) 87:8923-8927; and primer-guided nucleotide incorporation procedures by Prezant et al. (1992) Hum. Mutat. 1: 159-164; Ugozzoli et al. (1992) GATA 9:107-112; Nyreen et al. (1993) Anal. Biochem. 208:171-175.

Genomic DNA was fragmented using restriction endonucleases, and then subjected to one round of whole-genome amplification. The resulting fragments were then labeled and hybridized to an Affymetrix SNP chip, which contains probes for 25-mers surrounding known SNPs (approximately 100,000 for the Map 100 k chip set; approximately 500,000 for the Map500 k chip set). The chip was then scanned to retrieve intensities, which are then converted into genotype and genomic copy number calls using available software programs, including CNAT and dChip.

Table 3 summarizes genes that have increased gene amplification in BT474EEI cells as compared to BT474 or BT474JB cells.

TABLE 3
Genes amplified in BT-474 EEI: SNP analysis

1.1 VAV3 oncogene
1.3 NRAS (✓)
1.3 BCAS2 (✓)
1.3 PTPN22 (✓)
1.4 TGFB2
1.4 ESRRG
2.1 EPHA4 ((✓m EU vs. PAR only)
5.2 Oncostatin M R (✓)
5.2 FGF10 (✓, Ei vs. JB)
12.2 DYRK2
12.5 PPFIA2 (PTPRF)
12.5 NTS
15.1 FGF7
15.1 CYP19A1 (✓)
15.1 MAPK6 (✓)
15.1 BCL2-10 (✓)
17.1 BCAS3 (✓)
17.2 MAP2K6
(✓ = increased expression by Affy)

In addition, the following regions of chromosome was found to be lost in BT-474EEI cells.

1.1 TP73;

3.1 PKC delta

3.3 DAGK gamma

4.1 Caspase 3

8.2 NRG1

11.1 MMP26

18.1 PIK3C3

18.4 BCL-2

20.1 PCNA

22.1 PIM3

2.1 5HTR 2B

11.3 5hTR 3A and 3B

3.1 integrin 9-alpha

3.1 Activin A RIIB

4.1 IRF2

### Example 8

### Effects of Humanized Anti-IGF-1R Antibody 10H5 on BT-474 EEI Cell Growth

Since BT-474 EEI cells show upregulated expression of IGF-1R and estrogen receptor (ER) compared to the parental line, the effects of humanized anti-IGF-1R antibody 10H5 on BT-474 EEI growth were further examined.

BT-474 EEI cells were plated at a density of 5,000 cells per well in black 96-well microtiter plates in culture medium (high glucose DMEM:Ham's F-12 (50:50)+10% FBS+2 mM L-glutamine) and allowed to adhere overnight. The following day, the medium was removed and replaced with phenol red-free 50:50+10% charcoal-stripped FBS+2 mM L-glutamine containing different concentrations of the humanized anti-IGF-1R antibody 10H5 (Genentech, Inc.). Cells were incubated for 5 days and proliferation was measured using Celltiter-Glo (Promega Corp.). (Phenol red-free medium containing charcoal-stripped FBS provides steroid hormone-free conditions).

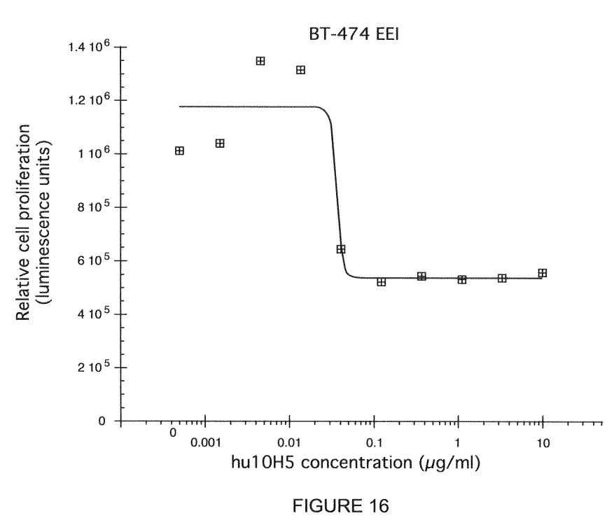

FIG. 16 shows inhibition of BT-474 EEI cell growth by humanized anti-IGF-1R antibody 10H5. Cell proliferation of BT-474 EEI substantially decreased upon treatment with, for example, 1 μg/ml, humanized anti-IGF-1R antibody 10H5. As previously shown, treatment of BT-474 EEI cells in regular culture medium showed no anti-proliferative effect of 10H5. However, when cells were grown under steroid-free conditions as in this Example, signaling through ER, which provides growth signals to the cells, was effectively eliminated. Under these conditions, proliferation of BT-474 EEI was reduced after treatment with 10H5.

### Example 9

### Synergistic Effects of Humanized Anti-IGF-1R Antibody Hu10H5 and Anti-Estrogen ICI 182,780 (Faslodex) on BT-474 EEI Cell Growth

In order to confirm the results of Example 8, the synergistic effects on BT-474 EEI cell growth of humanized anti-IGF-1R antibody hu10H5 and anti-Estrogen ICI 182,780 (Faslodex) were investigated. The study was performed in regular culture medium to provide the cells with a source of steroid hormones in order for Faslodex to work. BT-474 EEI cells were plated as described above (in Example 8). After allowing the cells to adhere overnight, the medium was removed and replaced with fresh culture medium (50:50+10% FBS+L-glutamine) containing a fixed dose (2 mg/ml) of 10H5 in combination with different concentrations of ICI 182,780 (Tocris Bioscience), a pure anti-estrogen (also known as Faslodex). Cells were treated for 5 days and proliferation was measured using Celltiter-Glo. Cells from passage 3 and passage 23 were tested with similar results.

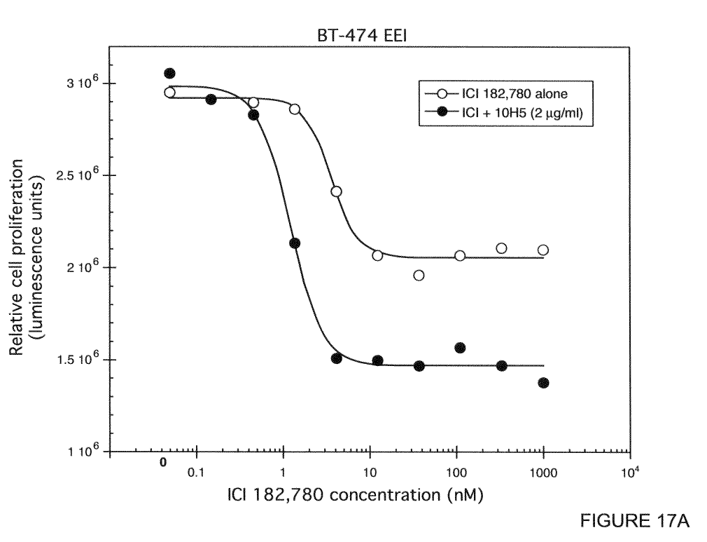

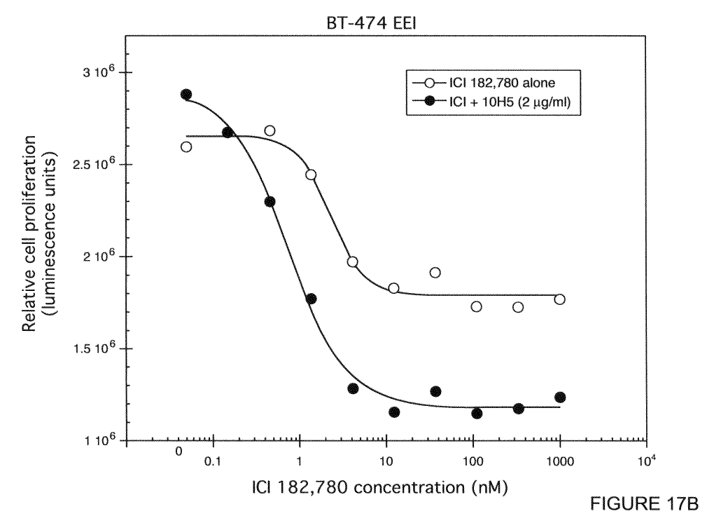

FIG. 17 shows that treatment of BT-474 EEI cells with the anti-IGF-1R antibody hu10H5 and anti-Estrogen ICI 182,780 (Faslodex) resulted in synergistic growth inhibition of BT-474 EEI cells. This study confirms the results in FIG. 16, suggesting that blockade of both IGF-1R and ER signaling pathways results in synergistic growth inhibition.

All references cited throughout the disclosure, and references cited therein, are hereby expressly incorporated by reference.

While the present invention is described with reference to certain embodiments, the invention is not so limited. One skilled in the art will appreciate that various modifications are possible without substantially altering the invention. All such modifications, which can be made without undue experimentation, are intended to be within the scope of the invention.

What is claimed is:

1. A BT-474-based stable breast cancer cell line that (1) overexpresses HER2 at a 3+ level or above; (2) is non-reliant on estrogen supplementation for in vivo growth, and (2) does not respond or responds poorly to treatment with trastuzumab.

2. The cell line of claim 2 which overexpresses HER2 at a 3+ level.

3. The cell line of claim 1 which is the Exogenous Estrogen Independent breast cancer cell line BT-474EEI.

4. The cell line of claim 3 which is growth inhibited by a trastuzumab-cytotoxic agent conjugate.

5. The cell line of claim 4 wherein the conjugate is a trastuzumab-auristatin or a trastuzumab-DM1 conjugate.

6. The cell line of any one of claims 1 to 5, which is immortalized.

7. The cell line of claim 1 obtained by multiple passages as xenografts in vivo and by intermittent in vivo culturing of a BT-474 human mammary adenocarcinoma cell line, and establishing a cell line from a transplanted tumor.

8. A model of HER2 overexpressing tumor that does not respond or responds poorly to treatment with trastuzumab, comprising the cell line of any one of claims 1-7.

9. A non-human animal model of HER2 overexpressing tumor that does not respond or responds poorly to treatment with trastuzumab comprising a non-human mammal inoculated with cells of the cell line of any one of claims 1-7.

10. The non-human animal model of claim 9 wherein said non-human animal is immunocompromised.

11. The non-human animal model of claim 6 wherein the immunocompromised non-human animal is a rodent.

12. The non-human animal model of claim 11 wherein said rodent is a mouse.

13. The non-human animal model of claim 12 wherein the cells are injected into the mammary fat pad of said mouse.

14. A method for identifying an agent for the treatment of a HER2 overexpressing tumor that does not respond or responds poorly to treatment with trastuzumab, comprising administering to a non-human animal carrying a BT-474-based tumor that: (1) overexpresses HER2 at a 3+ level or above; (2) is non-reliant on estrogen supplementation for in vivo growth, and (2) does not respond or responds poorly to treatment with trastuzumab, a candidate agent, and assessing tumor growth in said non-human animal, wherein inhibition of tumor growth compared to a control, non-treated non-human animal is indicative of the candidate being an agent for the treatment of HER2 overexpressing ligand activated tumor.

15. The method of claim 14 wherein said non-human animal is a rodent.

16. The method of claim 15 wherein said rodent is a mouse.

17. The method of claim 14 wherein said candidate agent is selected from the group consisting of polypeptides, antibodies, antibody fragments, antibody-cytotoxic agent conjugates, and peptide and non-peptide small molecules.

18. The method of claim 17 wherein said tumor is breast cancer.

19. A method for identifying an agent for the treatment of HER2 overexpressing tumor that does not respond or responds poorly to treatment with trastuzumab, comprising contacting culture of a cell line of claim 1 with a candidate agent, and assessing the growth of said cell line, wherein inhibition of growth compared to a control, is indicative of the candidate being an agent for the treatment of said HER2 overexpressing.

20. The method of claim 14 or claim 19 further comprising the step of treating a patient diagnosed with a HER2 overexpressing tumor that does not respond or responds poorly to treatment with trastuzumab, with the agent identified.

21. The method of claim claim 20 wherein said tumor is breast cancer.

22. A method of identifying an agent for increasing responsiveness to trastuzumab of a HER2 overexpressing tumor that does not respond or responds poorly to treatment with trastuzumab, comprising: administering to a non-human animal carrying a BT474-based tumor that: (1) overexpresses HER2 at a 3+ level or above; (2) is non-reliant on estrogen supplementation for in vivo growth, and (3) does not respond or responds poorly to treatment with trastuzumab, a candidate agent in the presence of trastuzumab; and assessing tumor growth in said non-human animal, wherein inhibition of tumor growth compared to a control, non-treated non-human animal is indicative of the candidate being an agent for the treatment of HER2 overexpressing ligand activated tumor when used in combination with trastuzumab.

23. The method of claim 22 wherein said non-human animal is a rodent.

24. The method of claim 23 wherein said rodent is a mouse.

25. The method of claim 24 wherein said candidate agent is selected from the group consisting of polypeptides, antibodies, antibody fragments, antibody-cytotoxic agent conjugates, and peptide and non-peptide small molecules.

26. The method of claim 25 wherein said tumor is breast cancer.

27. A method for identifying an agent for the treatment of HER2 overexpressing tumor that does not respond or responds poorly to treatment with trastuzumab, comprising contacting culture of a cell line of claim 1 with a candidate agent in the presence of trastuzumab and assessing the growth of said cell line, wherein inhibition of growth compared to a control, is indicative of the candidate being an agent for the treatment of said HER2 overexpressing when used in combination with trastuzumab.

28. The method of claim 14 or claim further comprising the step of treating a patient diagnosed with a HER2 overexpressing tumor that does not respond or responds poorly to treatment with trastuzumab, with the agent identified in combination with trastuzumab.

29. The method of claim 20 wherein said tumor is breast cancer.


SEQUENCE LISTING (39 sequences)

SEQ ID NO: 1 - DNA, 24 bp, Artificial Sequence
  Description of Artificial Sequence Synthetic primer
gccttgagtcatctattcaagcac

SEQ ID NO: 2 - DNA, 21 bp, Artificial Sequence
  Description of Artificial Sequence Synthetic primer
tgctactgtcattcgcacctg

SEQ ID NO: 3 - DNA, 25 bp, Artificial Sequence
  Description of Artificial Sequence Synthetic probe
agctctggccacaacagggcatttt

SEQ ID NO: 4 - DNA, 20 bp, Artificial Sequence
  Description of Artificial Sequence Synthetic primer
tctggacgtgccagtgtgaa

SEQ ID NO: 5 - DNA, 20 bp, Artificial Sequence
  Description of Artificial Sequence Synthetic primer
tgctccctgaggacacatca

SEQ ID NO: 6 - DNA, 24 bp, Artificial Sequence
  Description of Artificial Sequence Synthetic probe
cagaaggccaagtccgcagaagcc

SEQ ID NO: 7 - DNA, 24 bp, Artificial Sequence
  Description of Artificial Sequence Synthetic primer
ttctctactctaccattgcccaac

SEQ ID NO: 8 - DNA, 24 bp, Artificial Sequence
  Description of Artificial Sequence Synthetic primer
caccactatctcagcatctcggtc

SEQ ID NO: 9 - DNA, 24 bp, Artificial Sequence
  Description of Artificial Sequence Synthetic probe
acaccaactccagccacgctctgc

SEQ ID NO: 10 - DNA, 24 bp, Artificial Sequence
  Description of Artificial Sequence Synthetic primer
gagataaccagcattgagcacaac

SEQ ID NO: 11 - DNA, 24 bp, Artificial Sequence
  Description of Artificial Sequence Synthetic primer
agaggcaggtaacgaaactgatta

SEQ ID NO: 12 - DNA, 25 bp, Artificial Sequence
  Description of Artificial Sequence Synthetic probe
cctctccttcctgcggtctgttcga

SEQ ID NO: 13 - DNA, 21 bp, Artificial Sequence
  Description of Artificial Sequence Synthetic primer
agctaacccattatggcaaca

SEQ ID NO: 14 - DNA, 23 bp, Artificial Sequence
  Description of Artificial Sequence Synthetic primer
agttttcactgagtcagctccat

SEQ ID NO: 15 - DNA, 19 bp, Artificial Sequence
  Description of Artificial Sequence Synthetic probe
agggccctggacccaccac

SEQ ID NO: 16 - DNA, 18 bp, Artificial Sequence
  Description of Artificial Sequence Synthetic primer
ggacagcactgccagaga

SEQ ID NO: 17 - DNA, 22 bp, Artificial Sequence
  Description of Artificial Sequence Synthetic primer
caggtgattacaggccaagtag

SEQ ID NO: 18 - DNA, 22 bp, Artificial Sequence
  Description of Artificial Sequence Synthetic probe
cctgggtgtgccacagaccttc

SEQ ID NO: 19 - DNA, 20 bp, Artificial Sequence
  Description of Artificial Sequence Synthetic primer
tggctgacagcaggactaac

SEQ ID NO: 20 - DNA, 17 bp, Artificial Sequence
  Description of Artificial Sequence Synthetic primer
ctggcctggatttcttc

SEQ ID NO: 21 - DNA, 21 bp, Artificial Sequence
  Description of Artificial Sequence Synthetic probe
cagcaggccgcttctcgacac

SEQ ID NO: 22 - DNA, 24 bp, Artificial Sequence
  Description of Artificial Sequence Synthetic primer
atatcacattggagtcactgccca

SEQ ID NO: 23 - DNA, 23 bp, Artificial Sequence
  Description of Artificial Sequence Synthetic primer
gggtccattgtcttatgatccac

SEQ ID NO: 24 - DNA, 30 bp, Artificial Sequence
  Description of Artificial Sequence Synthetic probe
agccataaatgatgagtcggtcctctttcc

SEQ ID NO: 25 - DNA, 26 bp, Artificial Sequence
  Description of Artificial Sequence Synthetic primer
gaaagacttccatctagtcacaaaga

SEQ ID NO: 26 - DNA, 18 bp, Artificial Sequence
  Description of Artificial Sequence Synthetic primer
gggaggcccaatcctaga

SEQ ID NO: 27 - DNA, 20 bp, Artificial Sequence
  Description of Artificial Sequence Synthetic probe
tccttcgtccccagttgccg

SEQ ID NO: 28 - DNA, 19 bp, Artificial Sequence
  Description of Artificial Sequence Synthetic primer
tgccccaagcaatacaagc

SEQ ID NO: 29 - DNA, 16 bp, Artificial Sequence
  Description of Artificial Sequence Synthetic primer
cgtctgctcggccacc

SEQ ID NO: 30 - DNA, 27 bp, Artificial Sequence
  Description of Artificial Sequence Synthetic probe
aagcggcatctccctttgatgcagtaa

SEQ ID NO: 31 - DNA, 31 bp, Artificial Sequence
  Description of Artificial Sequence Synthetic primer
tgcatgcaatttaaagtaacttatttgacta

SEQ ID NO: 32 - DNA, 29 bp, Artificial Sequence
  Description of Artificial Sequence Synthetic primer
atcttaaggtacacaattatcaaagctga

SEQ ID NO: 33 - DNA, 36 bp, Artificial Sequence
  Description of Artificial Sequence Synthetic probe
tcggattactgaattgtatcaatttgtttgtgttca

SEQ ID NO: 34 - DNA, 20 bp, Artificial Sequence
  Description of Artificial Sequence Synthetic primer
agacggaccaaagccacttg

SEQ ID NO: 35 - DNA, 23 bp, Artificial Sequence
  Description of Artificial Sequence Synthetic primer
ccccgtgatgtaatacttttgca

SEQ ID NO: 36 - DNA, 24 bp, Artificial Sequence
  Description of Artificial Sequence Synthetic probe
ccactgcgggctctacttcatcgc

SEQ ID NO: 37 - DNA, 21 bp, Artificial Sequence
  Description of Artificial Sequence Synthetic primer
ttccctgtggcacttgacatt

SEQ ID NO: 38 - DNA, 22 bp, Artificial Sequence
  Description of Artificial Sequence Synthetic primer
cttttgcctctggcagtactca

SEQ ID NO: 39 - DNA, 35 bp, Artificial Sequence
  Description of Artificial Sequence Synthetic probe
tgtcttaaagtttttgaagtacatcttctggcccc